(19) United States
(12) Patent Application Publication
(10) Pub. No.: US 2025/0329442 A1
(43) Pub. Date: Oct. 23, 2025

(54) INFORMATION PROCESSING SYSTEM AND INFORMATION PROCESSING METHOD

(71) Applicant: PARAMOUNT BED CO., LTD., Tokyo (JP)

(72) Inventors: Yuichi WARAGAI, Tokyo (JP); Akio Shimomura, Tokyo (JP); Yoshitomo Nagamine, Tokyo (JP)

(21) Appl. No.: 19/037,807

(22) Filed: Jan. 27, 2025

(30) Foreign Application Priority Data
Apr. 22, 2024 (JP) 2024-068822

(51) Int. Cl. G16H 40/20; G16H 10/60; G16H 80/00

(52) U.S. Cl. CPC G16H 40/20; G16H 10/60; G16H 80/00

(57) ABSTRACT

An information processing system includes: a controller configured to set a condition for executing a notification processing related to a patient to a caregiver, the controller being configured to execute the notification processing if the condition is satisfied; and a display configured to display a setting screen used for setting of the notification processing by controlling the controller. The controller is configured to: obtain first information indicating a degree of loads needed for the caregiver to deal with notification processing on per-hospital ward basis, if the notification processing is executed, and obtain second information indicating a degree of allowable loads, based on third information indicating a target time in dealing with the notification processing and the number of the caregivers to deal with the notification processing, and the display is configured to display information indicating the relationship between the first information and the second information in the setting screen.

### BACKGROUND OF THE INVENTION

### Field

The present disclosure relates to an information processing system, an information processing method, and the like.

### Background

Heretofore, a method for obtaining a load at the time of caring for a care subject such as a patient has been known. For example, JP2021-197075A discloses a method for obtaining a practical workload for each staff.

### BRIEF DESCRIPTION OF THE DRAWINGS

FIG. 1 is a diagram illustrating a configuration example of an information processing system.

FIG. 2 is a diagram illustrating a functional configuration example of the information processing system.

FIG. 3 is a diagram illustrating a configuration example of a bed.

FIG. 4 is a diagram illustrating a configuration example of a detection device.

FIG. 5 is a flowchart illustrating notification setting processing in the information processing system.

FIG. 6 is a flowchart illustrating notification processing in the information processing system.

FIG. 7 is a flowchart illustrating processing of obtaining hospital ward load information.

FIG. 8 illustrates an example of load master data.

FIG. 9 illustrates an example of hospital room master data.

FIG. 10 illustrates an example of determination master data.

FIG. 11 illustrates an example of determination master data.

FIG. 12A illustrates an example of pictograms associated with pictogram codes.

FIG. 12B illustrates an example of pictograms associated with pictogram codes.

FIG. 13 illustrates an example of determination master data.

FIG. 14 illustrates an example of determination master data.

FIG. 15 is a flowchart illustrating processing of obtaining allowable load information.

FIG. 16 is a diagram illustrating processing of obtaining allowable load information by regression analysis.

FIG. 17 illustrates an example of a setting screen.

FIG. 18 illustrates an example of a patient screen.

FIG. 19 illustrates another example of the setting screen.

FIG. 20 illustrates a screen example for associating pictogram codes with a patient.

### DETAILED DESCRIPTION

One or more embodiments are now described with reference to the drawings, wherein like reference numerals are used to refer to like elements throughout. In the following description, for purposes 41 explanation, of numerous specific details are set forth in order to provide a thorough understanding of the various embodiments. It is evident, however, that the various embodiments can be practiced without these specific details (and without applying to any particular networked environment or standard).

As used in this disclosure, in some embodiments, the terms “component”, “system” and the like are intended to refer to, or comprise, a computer-related entity or a entity related to an operational apparatus with one or more specific functionalities, wherein the entity can be either hardware, or a combination of hardware and software in execution.

One or more components may reside within a process and/or thread of execution and a component may be localized on one computer and/or distributed between two or more computers. In addition, these components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software application or firmware application executed by a processor, wherein the processor can be internal or external to the apparatus and executes at least a part of the software or firmware application. As yet another example, a component can be an apparatus that provides specific functionality through electronic components without mechanical parts, the electronic components can comprise a processor therein to execute software stored on a non-transitory electronic memory or firmware that confers at least in part the functionality of the electronic components. While various components have been illustrated as separate components, it will be appreciated that multiple components can be implemented as a single component, or a single component can be implemented as multiple components, without departing from example embodiments. Further, the various embodiments can be implemented as a method, apparatus or article of manufacture using standard programming and/or engineering techniques to produce software, firmware, hardware or any combination thereof to control a computer to implement the disclosed subject matter. The term “article of manufacture” as used herein is intended to encompass a computer-readable (or machine-readable) device or computer-readable (or machine-readable) storage/communications media having a computer program stored thereon. For example, computer readable storage media can comprise, but are not limited to, magnetic storage devices (e.g., hard disk, floppy disk, magnetic strips), optical disks (e.g., compact disk (CD), digital versatile disk (DVD)), smart cards, and flash memory devices (e.g., card, stick, key drive). Of course, those skilled in the art will recognize many modifications can be made to this configuration without departing from the scope or spirit of the various embodiments.

In addition, the words “example” and “exemplary” are used herein to mean serving as an instance or illustration. Any embodiment or design described herein as “example” or “exemplary” is not necessarily to be construed as preferred or advantageous over other embodiments or designs. Rather, use of the word example or exemplary is intended to present concepts in a concrete fashion. As used in this application, the term “or” is intended to mean an inclusive “or” rather than an exclusive “or”. That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Embodiments described herein can be exploited in substantially any wireless communication technology, comprising, but not limited to, wireless fidelity (Wi-Fi), global system for mobile communications (GSM), universal mobile telecommunications system (UMTS), worldwide interoperability for microwave access (WiMAX), enhanced general packet radio service (enhanced GPRS), third generation partnership project (3GPP) long term evolution (LTE), third generation partnership project 2 (3GPP2) ultra mobile broadband (UMB), high speed packet access (HSPA), Z-Wave, Zigbee and other 802.XX wireless technologies and/or legacy telecommunication technologies.

In general, one aspect of the present application is an information processing system including:
a controller configured to set a condition for executing a notification processing related to a patient to a caregiver, the controller being configured to execute the notification processing if the condition is satisfied; and
a display configured to display a setting screen used for setting of the notification processing by controlling the controller, wherein
the controller is configured to:
obtain a first information indicating a degree of loads needed for the caregiver to deal with the notification processing on per-hospital ward basis, if the notification processing is executed, and
obtain a second information indicating a degree of allowable loads, based on a third information indicating a target time in dealing with the notification processing and the number of the caregivers to deal with the notification processing, and
the display is configured to display information indicating relationship between the first information and the second information in the setting screen.

Another aspect of the present application is an information processing method including:
performing processing of displaying, on a display, a setting screen used for setting of notification processing;
setting a condition for executing the notification processing related to a patient to a caregiver;
executing the notification processing if the condition is satisfied;
in the setting of the condition,
obtaining a first information indicating a degree of loads needed for the caregiver to deal with the notification processing on per-hospital ward basis, if the notification processing is executed, and
obtaining a second information, indicating a degree of allowable loads, based on a third information indicating a target time in dealing with the notification processing and the number of the caregivers to deal with the notification processing; and
in the processing of displaying the setting screen,
performing processing of displaying the setting screen including information indicating relationship between the first information and the second information.

Hereinbelow, this embodiment will be described with reference to the drawings. Throughout the drawings, the same or similar components are assigned with the same reference signs, and a redundant description thereof will be omitted. Note that, this embodiment to be described below is not intended to unjustly limit the contents described in the scope of claims. In addition, not all configurations to be described in this embodiment are necessarily essential elements of the present disclosure.

### 1. System Configuration Example

An information processing system 10 according to this embodiment is a system designed to perform notification processing for a caregiver appropriately in a scene where the caregiver cares for a patient, for example. Specifically, the information processing system 10 suppresses cases such as a case where notifications are given to the caregiver excessively and a case where necessary notifications on a patient who needs care fail to be given to the caregiver. Processing in the information processing system 10 will be described in detail later. Note that, the patient in this embodiment is a patient who is hospitalized in a hospital, for example, but the patient may be a person who is living in a care facility. The caregiver is a nurse who works for a hospital in a narrow sense, but may include a work therapist and a physical therapist. The caregiver may also include a caregiver, a care manager, and the like.

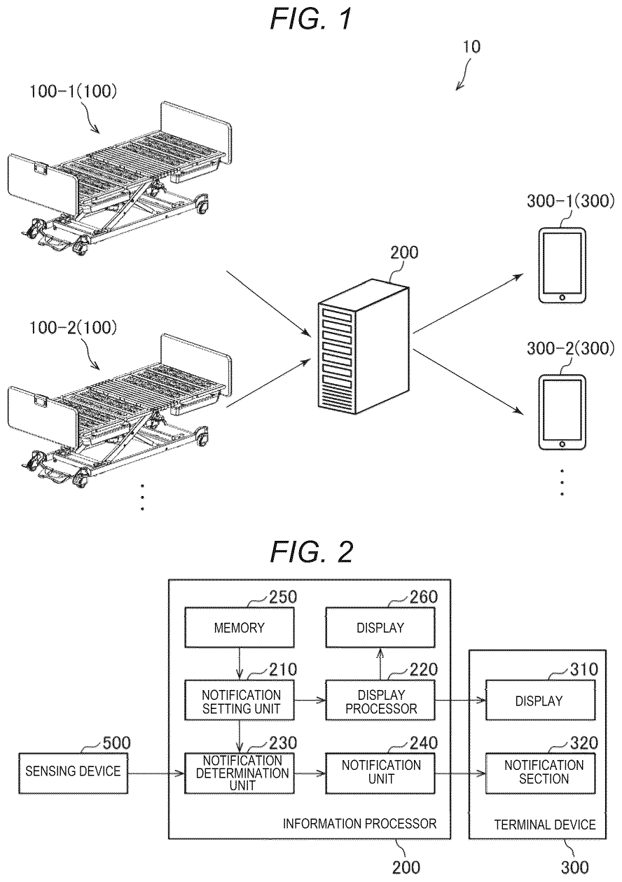

FIG. 1 is a diagram illustrating a configuration example of the information processing system 10 according to this embodiment. The information processing system 10 includes, for example: a bed 100; an information processor 200; and a terminal device 300. However, the configuration of the information processing system 10 is not limited to the example of FIG. 1, and can be modified such as omitting a part of the configuration or adding a different configuration. For example, the information processing system 10 according to this embodiment may be a part of the configuration illustrated in FIG. 1, or the information processing system 10 may be the information processor 200, for example. Further, although two beds 100-1 and 100-2 are illustrated as the bed 100 in FIG. 1, the number of beds included in the bed 100 is not limited to this. Likewise, although two terminal devices 300-1 and 300-2 are illustrated as the terminal device 300 in FIG. 1, the number of terminal devices included in the terminal device 300 is not limited to this. The information processing system 10 may also include a sensing device 500 such as a detection device 400 to be described later using FIG. 4.

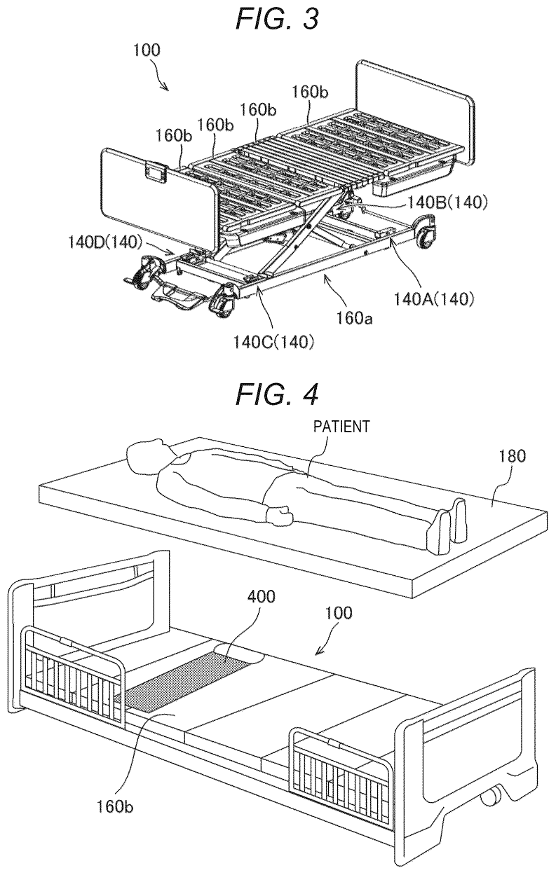

The bed 100 is bedding to be used by the patient. The bed 100 mentioned here may include movable parts that adjust the height and angle (such as a leg unit 160a and sections 160b to be described later using FIG. 3). The bed 100 may also include a load sensor that detects pressure (such as load sensors 140A to 140D to be described later using FIG. 3). The bed 100 outputs, to the information processor 200, information on the height and angle, a sensing result using sensors such as the load sensors 140A to 140D, and the like.

The information processor 200 is configured to perform processing on whether notification to the caregiver is necessary by determining the state of the patient based on information from the bed 100. The bed 100 and the information processor 200 communicate with each other by wireless using a method such as IEEE802.11 or Bluetooth. However, the bed 100 and the information processor 200 may communicate with each other by wire.

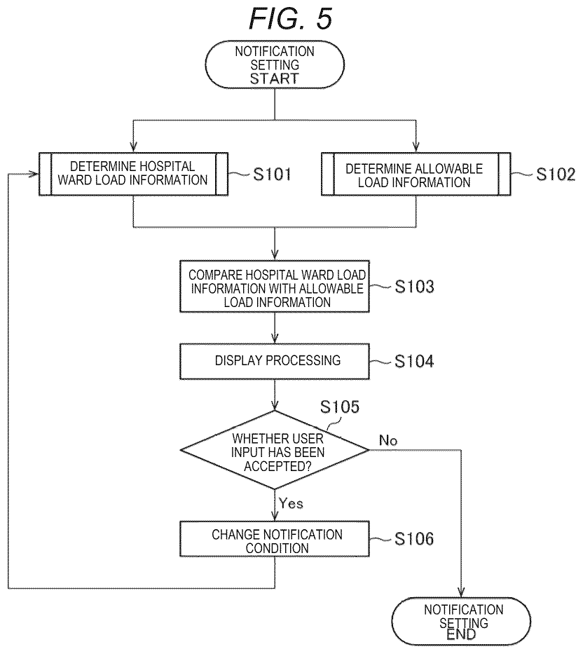

The information processor 200 is a server system, for example. The server system may be one server or may include multiple servers. For example, the server system may include a database server and an application server. The database server may store information that is transmitted from the bed 100. The application server performs various kinds of processing based on the information. The application server executes processing of various steps to be described later using FIG. 5, for example. Note that, the multiple servers mentioned here may be physical servers or may be virtual servers. In addition, in the case of using virtual servers, the virtual servers may be provided in one physical server or may be dispersed in multiple physical servers. As described above, the specific configuration of the server system of this embodiment can be modified in various ways.

For example, the information processor 200 is a management server provided in a hospital, and may perform various kinds of processing, such as storage of electronic medical records and management of patients and caregivers, in addition to notification processing for a caregiver. Alternatively, the information processor 200 may be a server system provided separately from the management server in the hospital. For example, the information processor 200 is a cloud server connected to a network of the hospital via a public communication network such as the Internet, and may receive information from the bed 100 via a management server not illustrated in FIG. 1. Alternatively, the information processor 200 may include both the management server and the cloud server in the hospital, and may be implemented by dispersed processing of both of these servers. Still alternatively, the information processor 200 may be implemented by a device of another mode such as a personal computer (PC).

The terminal device 300 is a device to be used by a caregiver, for example. Specifically, the terminal device 300 is a device supplied to each of multiple caregivers, and is configured to perform processing of presenting the caregiver with various kinds of information on a patient acquired from the information processor 200. Note that, the terminal device 300 and the bed 100 do not necessarily have to be provided separately. As will be described later, the terminal device 300 may be incorporated in the bed 100.

In this embodiment, conditions for executing notification processing on a patient are set, and the information processor 200 determines whether the execution conditions are met. If the execution conditions are met, the information processor 200 causes a notification section 320 (such as a speaker) of the terminal device 300 to execute the notification processing. This makes it possible to prompt a caregiver to do an appropriate action when an anomaly occurs in the patient or when the caregiver needs to watch over the patient.

FIG. 2 is a diagram illustrating the functional configuration of the information processing system 10. The information processing system 10 includes: the sensing device 500; the information processor 200; and the terminal device 300. The information processor 200 includes: a notification setting unit 210; a display processor 220; a notification determination unit 230; a notification unit 240; a memory 250; and a display 260. The terminal device 300 includes a display 310 and a notification section 320. However, the configuration of each of the devices of the information processing system 10 is not limited to the example of FIG. 2, and can be modified such as omitting a part of the configuration or adding a different configuration.

The sensing device 500 is configured to output, to the information processor 200, a sensing result which is a result of sensing of the state of a patient. The sensing device 500 mentioned here is the bed 100 including the load sensors 140A to 140D, for example. However, various devices can be employed as the sensing device 500, such as the detection device 400 to be described later using FIG. 4, an image sensor such as a camera, a wearable device, a radio frequency identification (RFID), a tag, and an RFID reader.

The sensing device 500 is not limited to a device that acquires information indicating the state of a patient, but may also acquire information indicating the state of the patient's surrounding environment. For example, in a case where the bed 100 is used as the sensing device 500, the sensing result of the bed 100 may include information such as the height of the bed and the angle of each section 160b. Various devices such as a thermometer, a hygrometer, and a luminometer can be employed as the sensing device 500 that senses the state of surrounding environment. The sensing device 500 will be described in detail later.

The notification setting unit 210 of the information processor 200 is configured to perform processing of setting conditions for executing notification processing that notifies a caregiver of necessity to check a patient (such conditions are hereinafter referred to as notification conditions). For example, the notification conditions may be information indicating the position or posture of a patient around the bed 100, such as “bed departure” and “edge sitting position”. The term “bed departure” indicates a condition for executing notification processing when the patient departs from the bed 100. The term “edge sitting position” indicates a condition for executing notification processing when the patient takes a posture of sitting at an edge part of the bed 100. The notification conditions based on the position/posture of the patient (hereinafter also referred to as patient notification conditions) are not limited to the “bed departure” and the “edge sitting position”. The notification conditions may also include conditions other than the patient notification conditions. The notification conditions will be described in detail later.

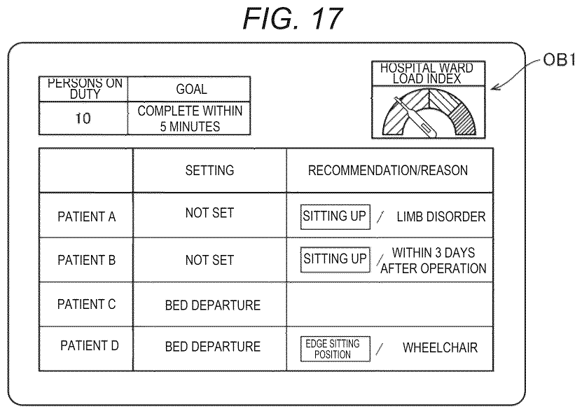

The display processor 220 is configured to perform processing of displaying, on the display, a setting screen which is a screen for setting the notification conditions. The setting screen will be described in detail later using FIG. 17. Note that, the display mentioned here may be the display 260 of the information processor 200 or may be the display 310 of the terminal device 300. For example, in a case where a user of the information processor 200 (such as a supervisor of a hospital) sets the notification conditions, the display processor 220 displays the setting screen on the display 260. Meanwhile, in a case where a caregiver sets the notification conditions, the display processor 220 displays the setting screen on the display 310. The display processor 220 may also perform processing of displaying the setting screen on a display other than the display 260 and the display 310.

The notification determination unit 230 is configured to acquire a sensing result from the sensing device 500 via a communicator (communication interface) (not illustrated), for example. The notification determination unit 230 is then configured to determine whether the notification conditions are met based on the sensing result.

The notification unit 240 is configured to execute notification processing for a caregiver if the notification determination unit 230 determines that the notification conditions are met. For example, the notification unit 240 causes the notification section 320 of the terminal device 300 to execute notification processing. When multiple caregivers have the terminal devices 300 respectively, the notification unit 240 may extract a caregiver who is supposed to be in charge of a target patient among the multiple caregivers, and cause the notification section 320 of the terminal device 300 of the caregiver thus extracted to execute the notification processing. Alternatively, the notification unit 240 may cause the multiple terminal devices 300 to execute the notification processing collectively.

The notification setting unit 210, the display processor 220, the notification determination unit 230, and the notification unit 240 described above may be included in a processing unit (not illustrated). The processing unit in the embodiment is configured with the following hardware. The hardware can include at least one of a circuit that processes a digital signal and a circuit that processes an analog signal. For example, the hardware may include one or multiple circuit devices and one or multiple circuit elements mounted on a circuit board. One or multiple circuit devices are, for example, an integrated circuit (IC) and a field-programmable gate array (FPGA). Examples of the one or multiple circuit elements include a resistor and a capacitor.

Alternatively, the processing unit may be implemented by the following processor. The information processor 200 of this embodiment includes a memory that stores information and a processor that operates based on information stored in the memory. For example, the information is a program, various kinds of data, and the like. The memory may be the memory 250, or may be another memory. The processor includes hardware. As the processor, various processors including a central processing unit (CPU), a graphics processing unit (GPU), a digital signal processor (DSP), and the like can be used. The memory may be a semiconductor memory such as a static random access memory (SRAM), a dynamic random access memory (DRAM), and a flash memory, may be a register, may be a magnetic storage device such as a hard disk device (HDD: hard disk drive), and may be an optical storage device such as an optical disc device. For example, the memory stores a computer readable command, and the functions of the units of the processing unit (the notification setting unit 210, the display processor 220, the notification determination unit 230, and the notification unit 240) are implemented as processing by causing the processor to execute the command. The command mentioned here may be a command of a command set constituting a program or may be a command that gives operation instructions to a hardware circuit of the processor.

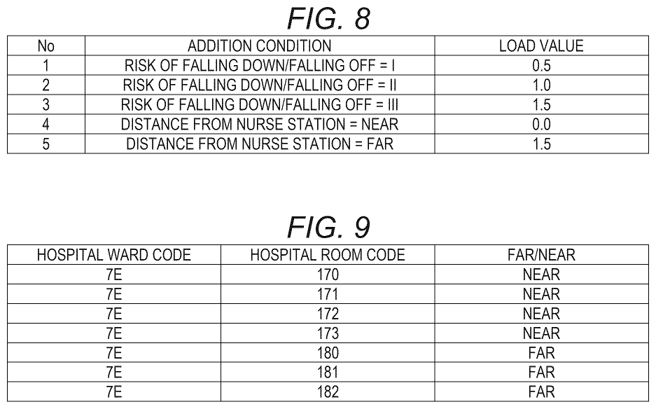

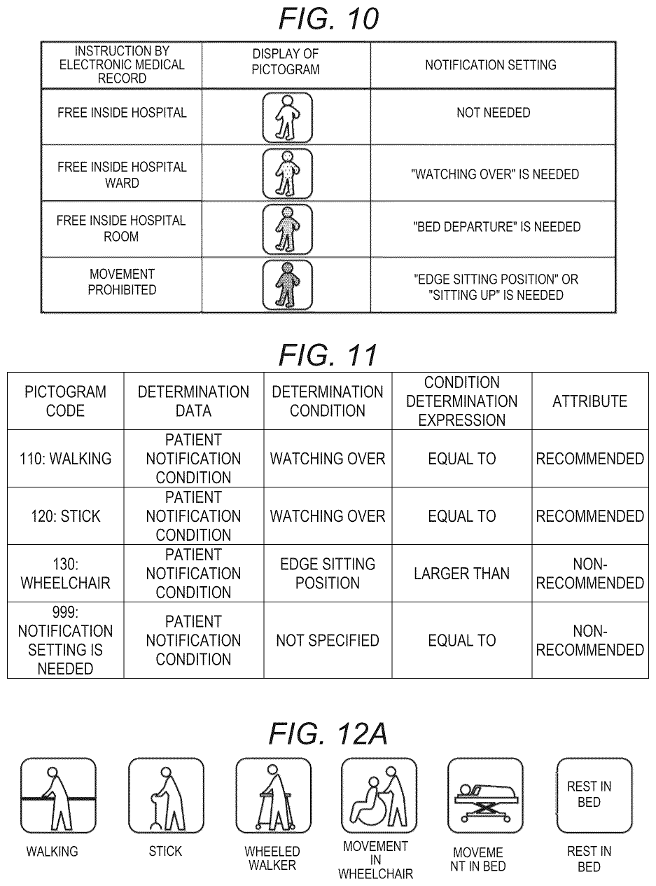

The memory 250 is a work area of the units of the processing unit, and stores various kinds of information. The memory 250 can be implemented by various memories, and the memory may be a semiconductor memory such as SRAM, DRAM, a read only memory (ROM), and a flash memory, may be a register, may be a magnetic storage device, and may be an optical storage device. The memory 250 may store the notification conditions for each patient and the sensing result from the sensing device 500, for example. The memory 250 may also store load master data to be described later using FIG. 8, hospital room master data to be described later using FIG. 9, and determination master data to be described later using FIGS. 10 and 11 and FIGS. 13 and 14. The memory 250 may also store a patient's profile, an electronic medical record, and caregiver management information (such as work shifts and skill information).

The display 260 is an interface that displays various kinds of information, and may be a liquid crystal display, may be an organic EL display, or may be a display using another method.

Likewise, the display 310 of the terminal device 300 is an interface that displays various kinds of information, and may be a liquid crystal display, may be an organic EL display, or may be a display using another method.

The notification section 320 is configured to notify a user of the terminal device 300 of information. The notification section 320 may be a speaker that gives notification by sound, for example. Alternatively, the notification section 320 may be a vibrator (motor) that gives notification by vibration. Still alternatively, the display 310 may also function as the notification section 320. In this case, the display 310 being the notification section 320 gives notification by an image.

As has been described above, the information processing system 10 (the information processor 200 in a narrow sense) of this embodiment includes: the notification setting unit 210; the notification unit 240; and the display processor 220. The notification setting unit 210 of this embodiment obtains hospital ward load information and allowable load information. The hospital ward load information is information indicating the degree of loads needed for a caregiver to deal with the notification processing on per-hospital ward basis, when notification processing on a patient in a hospital ward to be subjected to processing is performed. The allowable load information is information indicating the degree of allowable loads, and is obtained based on goal information indicating a time goal in dealing with the notification processing and the number of caregivers to deal with the notification processing. The display processor 220 performs processing of displaying a setting screen including information on the relationship between the hospital ward load information and the allowable load information. The hospital ward load information and the allowable load information will be described in detail later. Also, the setting screen will be described later using FIG. 17.

According to the method of this embodiment, it is possible to calculate, as hospital ward load information, a load on a caregiver on per-hospital ward basis that is presumed based on notification conditions having been set, and calculate, as allowable load information, an allowable load that is presumed based on target time and the number of caregivers. By displaying such relationship in the setting screen, it is possible to let a user (a supervisor and/or a caregiver) recognize whether the current notification conditions fall within an allowable load range.

Accordingly, if the hospital ward load information exceeds the allowable load information, the user is assumed to delete some of the notification conditions or change them to modes with less load, which can inhibit notifications from being given too frequently for the caregiver to take care of. Meanwhile, if the allowable load information exceeds the hospital ward load information, it means the caregiver still has room for additional care, so the user can add a new notification condition, for example. As a result, this can suppress failure to set notification conditions on a patient who highly needs to be taken care of. Note that, at the time of adding a notification condition, the display processor 220 may perform processing of displaying a setting screen including a notification condition recommended for a target patient. Details thereof will be described later.

A part or all of the processing that is performed by the information processing system 10 of this embodiment may be implemented by a program. The processing to be performed by the information processing system 10 is processing to be performed by the information processor 200 in a narrow sense, but may include processing to be executed by the bed 100 or the terminal device 300.

The program according to this embodiment can be stored, for example, in a non-transient information storage medium (information memory device) which is a computer-readable medium. The information storage medium can be implemented by an optical disc, a memory card, an HDD, or a semiconductor memory, for example. The semiconductor memory is a ROM, for example. The processing unit (the notification setting unit 210, the display processor 220, the notification determination unit 230, and the notification unit 240) and the like perform various kinds of processing of this embodiment based on the program stored in the information storage medium. In other words, the information storage medium stores the program for causing a computer to function as the processing unit. The computer is a device including an input device, a processing unit, a memory, and an output unit. Specifically, the program according to this embodiment is a program for causing the computer to execute steps which will be described later using figures such as FIG. 4.

The method in this embodiment can be applied to an information processing method including the following steps. The information processing method includes: a step of performing processing of displaying a setting screen, used for setting of notification processing, on a display; a step of setting conditions for executing notification processing that notifies a caregiver of necessity to check a patient; and a step of executing the notification processing if it is determined that the execution conditions are met. Then, in the information processing method, at the time of the setting of the execution conditions, hospital ward load information is obtained which indicates, when the notification processing on a patient is performed, the degree of loads needed for a caregiver to deal with the notification processing on per-hospital ward basis, allowable load information which indicates the degree of allowable loads is obtained based on goal information indicating a time goal in dealing with the notification processing and the number of caregivers to deal with the notification processing and, at the time of performing processing of displaying the setting screen, processing of displaying the setting screen including information on the relationship between the hospital ward load information and the allowable load information is performed.

### 2. Specific Example of Sensing Device

Next, a specific example of the sensing device 500 will be described. As has been described using FIG. 1, the sensing device 500 may be the bed 100.

FIG. 3 illustrates a specific configuration example of the bed 100. As illustrated in FIG. 3, the load sensors 140 that detect pressure are arranged in the bed 100. FIG. 3 illustrates an example in which the load sensors 140A to 140D are respectively arranged at four locations of the bed 100 (in a narrow sense, four locations corresponding to four corners of the bed 100). However, the number and positions of the load sensors 140 are not limited thereto, and various modifications can be made.

For example, each load sensor 140 may be a piezoelectric sensor using a piezoelectric substance that generates charges upon application of pressure, or may be a strain sensor that detects strain of a material due to pressure as a change in electric resistance. In addition, various methods including the piezoelectric sensor and the strain sensor described above have been known as the load sensor, and these methods can be widely employed in this embodiment. Accordingly, a description of the detailed configuration of the load sensor 140 will be omitted.

Meanwhile, the bed 100 may be a nursing care bed that includes the leg unit 160a and the sections 160b as movable parts 160 and that is capable of adjusting the height by moving the leg unit 160a and adjusting the angle (such as a back angle) by moving the sections 160b.

The bed 100 includes a driving unit (not illustrated), such as an actuator, that drives the movable parts 160 of the bed 100. For example, the bed 100 includes the multiple sections 160b sectioned as multiple members as the movable parts 160, and the driving unit adjusts the angle of the sections 160b by changing the position and posture of at least a part of the multiple sections 160b under control of a processor (not illustrated) included in the bed 100. For example, by driving of the driving unit, control over the back angle (control over the section 160b on a head part side) and control over a foot angle (control over the section 160b on a foot side) may be performed.

Meanwhile, the driving unit is an actuator or the like that drives the leg unit 160a. This makes it possible to adjust the total height of the bed 100 by driving of the driving unit.

The bed 100 may output outputs from the load sensors 140A to 140D to the information processor 200 as a sensing result on the position and posture of a patient, may output the state (the height and angle) of the movable parts 160 to the information processor 200 as a sensing result on patient's environment, or may perform both of these processing.

FIG. 4 is another example of the sensing device 500, and is a diagram illustrating the configuration of the detection device 400 that measures biological information. The detection device 400 is a sheet-shaped or plate-shaped device that is provided between the sections 160b of the bed 100 and a mattress 180, for example, as illustrated in FIG. 4. The detection device 400 is a device that senses information on patient's sleeping. The detection device 400 includes a pressure sensor (such as a pneumatic sensor) that outputs a pressure value.

The detection device 400 is configured to detect, when a user goes to bed, body vibration (body movement, vibration) of a user via the mattress 180. The detection device 400 obtains, based on the body vibration thus detected, information on a respiratory rate, a heartbeat rate, the amount of activity, a posture, awake/sleeping, and bed departure/bed presence. Note that, the detection device 400 may output sensor data indicating the body vibration, and another device such as the information processor 200 may execute processing of obtaining information such as the respiratory rate based on the sensor data. Hereinbelow, a description will be given of an example in which the detection device 400 outputs information such as the respiratory rate.

For example, the detection device 400 may analyze the periodicity of the body movement, and calculate a respiratory rate and a heartbeat rate from a peak frequency. The analysis of the periodicity is a Fourier transform, for example. The respiratory rate is the frequency of respiration per unit time. The heartbeat rate is the frequency of heartbeats per unit time. The unit time is one minute, for example. In addition, the detection device 400 may detect body vibration per sampling unit time and output the number of times of the body vibration thus detected as the amount of activity. Moreover, since a pressure value to be detected decreases in the bed departure of the user compared with in the bed presence, the detection device 400 may make determination of bed departure/bed presence based on the pressure value and a time-series change in the pressure value. However, the method of determination of bed departure/bed presence is not limited to this, and various modifications such as detection of vibration can be made. The detection device 400 may make determination of non REM sleep and REM sleep, and determination of a sleep depth. The determination on sleeping may be executed based on the respiratory rate and the heartbeat rate, may be executed based on the amount of body movement (such as the amount of activity), or may be executed using both of them.

The detection device 400 is configured to output biological information of a patient (information such as respiration, heartbeat, and sleeping) and information on the position and posture (information such as posture and bed departure/bed presence) to the information processor 200 as a sensing result. Note that, as described previously, in a case where the bed 100 includes the load sensors 140, it is possible to presume the position and posture of the patient based on information from the load sensors 140. In this case, any of the bed 100 and the detection device 400 does not necessarily have to output the sensing result on the position and posture of the patient. Alternatively, the information processor 200 may make determination on notification conditions by acquiring sensing results on the position and posture of the patient from both of the bed 100 and the detection device 400 and using these sensing results.

Meanwhile, the sensing device 500 is not limited to the above example. For example, the sensing device 500 may include cameras arranged at various positions of a hospital ward. The cameras are arranged in a room of a patient and shared spaces of the hospital ward (such as a corridor and a dayroom), for example, and may output image data to the information processor 200 as sensing results. The information processor 200 determines the position of the patient by subjecting the image data to image processing (person detection processing in a narrow sense). For example, the information processor 200 may identify the patient by person's face authentication processing. This enables the information processor 200 to presume the position of the patient based on which patient is captured in image data output from which camera. Methods of performing skeletal tracking based on image data have also been widely known, and in this embodiment, it is also possible to presume the posture of the patient based on camera output by employing such methods.

In addition, the sensing device 500 may include a wearable device. For example, devices capable of detecting a pulse rate and the like of a patient have been widely known as watch type wearable devices. By using such wearable devices as the sensing device 500, it is possible to output biological information of the patient to the information processor 200.

Further, the sensing device 500 may include an RFID tag that is worn by a patient and an RFID reader that is disposed at a predetermined position in a hospital. The memory 250 may store data that associates a patient ID with a tag ID of the RFID tag and data indicating the position where the RFID reader is disposed. This makes it possible to presume the position of the patient based on a result of reading the RFID reader.

Besides, the sensing device 500 may include a thermometer and a hygrometer which are devices for measuring environment information. The thermometer detects temperature in the surrounding of a patient. The hygrometer detects humidity in the surrounding of the patient. The thermometer and the hygrometer transmits the temperature and the humidity to the information processor 200 as a sensing result of the patient's surrounding environment.

The sensing device 500 that senses patient's surrounding environment may include a CO2 measuring device, a luminometer, and the like. In this case, the sensing device 500 transmits information such as carbon dioxide concentration and lighting intensity to the information processor 200 as a sensing result.

The sensing device 500 in this embodiment is not limited to those exemplified above, and may expand its range to various devices capable of sensing the state of a patient or the state of surrounding environment.

### 3. Notification Condition Setting and Notification Processing

A description will be given of a flow of processing to be executed by the information processing system 10 (the information processor 200 in a narrow sense). First of all, the overall flow will be described, and then each processing will be described in detail.

### 3.1 Overall Flow

FIG. 5 is a flowchart illustrating notification setting processing in the notification setting unit 210. Upon start of this processing, first, in Step S101, the notification setting unit 210 obtains hospital ward load information. The hospital ward load information is information obtained by obtaining the degree of loads needed for a caregiver to deal with notification processing on per-hospital ward basis.

Specifically, when notification processing on a patient is executed in Step S101, the notification setting unit 210 may calculate, as patient load information, information indicating the degree of loads needed for a caregiver to deal with the notification processing. In other words, the patient load information is information indicating a load on per-patient basis. The notification setting unit 210 obtains hospital ward load information based on pieces of patient load information of multiple patients who are hospitalized in a hospital ward. In addition, in Step S101, the notification setting unit 210 may perform processing of determining a notification condition recommended for the patient. The processing of Step S101 will be described in detail later.

Further, in Step S102, the notification setting unit 210 obtains information indicating the degree of allowable loads on per-hospital ward basis based on past records, and sets the information as allowable load information. For example, the notification setting unit 210 may obtain the allowable load information by obtaining the degree of loads allowable for each caregiver based on the past records and multiplying the obtained value by the number of caregivers actually on duty. The processing of Step S102 will be described in detail later.

The hospital ward load information and the allowable load information are obtained by the above processing. Note that, the processing of Step S101 and the processing of Step S102 do not necessarily have to be executed consecutively, and these processing may be executed independently. Further, the timing and frequency of executing the processing of Step S101 and the processing of Step S102 may be different from each other. For example, a trigger for executing the processing of Step S101 and a trigger for executing the processing of Step S102 may be different from each other. The timing of executing these processing will be described later.

Next, in Step S103, the notification setting unit 210 determines the magnitude relationship between the latest hospital ward load information and allowable load information based on processing of comparison between these pieces of information. For example, if a value of the hospital ward load information exceeds a value of the allowable load information, it is determined that notification setting tends to be excessive and it may take time for a caregiver to take care of. Meanwhile, if the value of the hospital ward load information is lower than the value of the allowable load information, it is determined that the caregiver still has room for additional care, so another notification condition can be added. Meanwhile, if the hospital ward load information and the allowable load information are of the same degree, it is determined that these are in a balanced state.

In Step S104, the display processor 220 performs processing of displaying, on the display 260, a setting screen reflecting the determination result in Step S103. Note that, as has been described above, the setting screen may be displayed on the display 310 of the terminal device 300 or may be a display of another device. For example, as will be described later using FIG. 17, the display processor 220 may display an object OB1 indicating the magnitude relationship between the hospital ward load information and the allowable load information. The setting screen will be described in detail later.

In addition, in Step S105, the notification setting unit 210 may determine whether an input operation of a user in the setting screen has been accepted. For example, in a case where a user input is made using a user interface device of a device that displays the setting screen, information indicating the user input is transmitted to the notification setting unit 210. The user input mentioned here includes addition, deletion, change, and the like of notification conditions, for example.

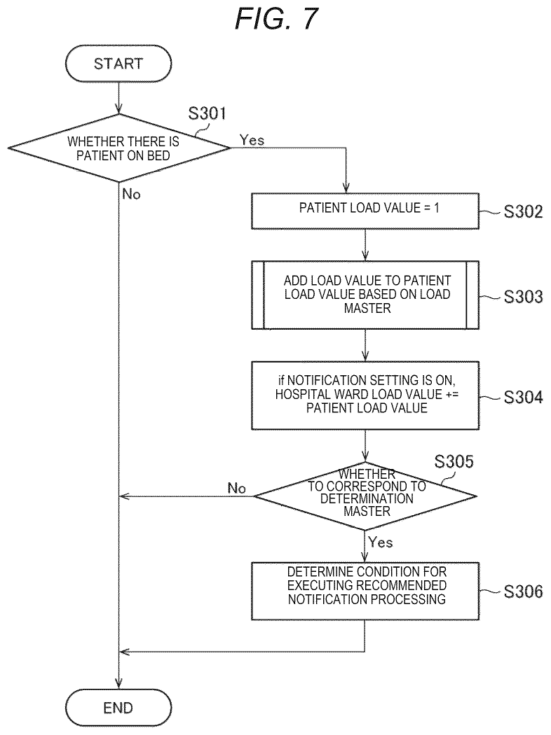

Upon acceptance of a user input (Step S105: Yes), in Step S106, the notification setting unit 210 performs processing of updating patient notification conditions based on the contents of the input operation. Note that, as will be described later using FIG. 7, a value of hospital ward load information may vary when the patient notification conditions are changed. Accordingly, after the processing of Step S106, the notification setting unit 210 may return to Step S101 to perform processing of updating the hospital ward load information.

If the setting screen is closed without acceptance of a user input (Step S105: No), the notification setting unit 210 ends the processing.

By performing the processing illustrated in FIG. 5, it is possible to prompt a user to do an action such as addition, deletion, and change of notification conditions while clearly specifying the relationship between loads, which might occur as a result of the set notification conditions, and loads that a caregiver can take care of. As a result, this can suppress cases such as a case where excessive notification setting is given and a case where necessary notification setting fails to be given.

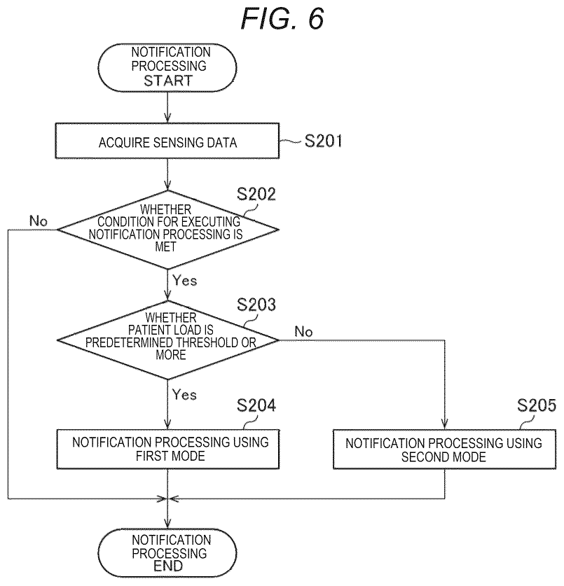

When notification setting processing is done, the information processing system 10 executes notification processing using the set notification conditions. FIG. 6 is a flowchart illustrating notification processing in the notification determination unit 230 and the notification unit 240. Upon start of this processing, first, in Step S201, the notification determination unit 230 acquires a sensing result from the sensing device 500. For example, each of the multiple sensing devices 500 transmits the sensing result at a frequency, set for each of these devices, to the notification determination unit 230.

In Step S202, the notification determination unit 230 determines, for each patient, whether the set notification conditions are met based on a sensing result. For example, the notification determination unit 230 may determine, upon acquisition of a sensing result for a predetermined period, whether the notification conditions are met based on the sensing result.

The notification conditions mentioned here may be patient notification conditions on the position, posture, sleeping/awake of a patient, for example. For example, the notification setting unit 210 may be capable of setting conditions such as “watching over”, “bed departure”, “edge sitting position”, “sitting up”, and “awake” as the patient notification conditions. The term “watching over” indicates a condition for executing notification processing when a predetermined period has passed since the patient goes outside his/her room. Here, the term “watching over” may be a condition for executing notification processing when a predetermined period has passed since the patient departs from the bed 100. The term “bed departure” indicates a condition for executing notification processing when the patient departs from the bed 100. The term “edge sitting position” indicates a condition for executing notification processing when the patient takes a posture of sitting at an edge part of the bed 100. The term “sitting up” indicates a condition for executing notification processing when the patient takes a posture of sitting up on the bed 100. The term “awake” indicates a condition for executing notification processing when the patient transitions from the sleeping state to the awake state.

As described above, by using the sensing device 500, it is possible to execute various kinds of processing including the detection of the position and posture of a patient, and determination on sleeping. Accordingly, the notification determination unit 230 executes, based on the sensing result, determination on whether each of the notification conditions including “watching over”, “bed departure”, “edge sitting position”, “sitting up”, and “awake” is met.

In addition, the notification conditions mentioned here may be conditions on patient's surrounding environment. For example, the notification setting unit 210 may be capable of setting conditions such as “bed height of X cm” and “bed angle of θ or more” as the notification conditions. The notification conditions on the bed 100 are hereinafter also referred to as a bed current state.

The term “bed height of X cm” indicates a condition for executing notification processing when the height of a surface of the section 160b (or a surface of the mattress 180) of the bed 100 is shifted from X cm. Note that, X mentioned here is not limited to a single numeric value and may be a given numeric range. Alternatively, multiple notification conditions may be used for the height of the bed 100 by preparing multiple values as X, for example, X1, X2, X3, The term “bed angle of θ or more” indicates a condition for executing notification processing when the angle of the section 160b of the bed 100 is θ (degrees) or more. As θ mentioned here, multiple values such as 30°, 45°, and 60° may be set. In addition, as the bed angle, a notification condition on the back angle and a notification condition on the foot angle may be set.

As has been described above, it is possible to obtain the bed height and the bed angle based on the state of the movable parts 160 of the bed 100 (the driving state of the driving unit), for example. Accordingly, the notification determination unit 230 executes, based on the sensing result from the bed 100, determination on whether each of the notification conditions including “bed height of X cm” and “bed angle of θ or more” is met.

Besides, as the notification conditions, biological information such as heartbeat may be used, environment information such as temperature, humidity, and lighting intensity may be used, or a combination of two or more of them may be used.

If determining that the notification conditions are met (Step S202: Yes), in Step S203, the notification determination unit 230 determines whether a value of patient load information of a target patient is a threshold or more. As described previously, the patient load information is information indicating the degree of loads needed for a caregiver to deal with the notification processing, when notification processing on a patient is executed, and this information is obtained in the processing of Step S101 in FIG. 5. Details of the processing of obtaining the patient load information will be described later using Steps S302 and S303 in FIG. 7.

If the patient load information is the threshold or more (Step S203: Yes), in Step S204, the notification unit 240 performs notification processing using a first mode. If the patient load information is below the threshold (Step S203: No), in Step S205, the notification unit 240 performs notification processing using a second mode different from the first mode. For example, when the notification section 320 of the terminal device 300 is a speaker that outputs sound, the notification unit 240 performs processing of causing the speaker to output first sound in Step S204 and performs processing of causing the speaker to output second sound in Step S205. Alternatively, when the notification section 320 is a vibrator (motor), the notification unit 240 may perform processing of vibrating the vibrator with a first vibration pattern in Step S204 and perform processing of vibrating the vibrator with a second vibration pattern in Step S205.

Meanwhile, when the display 310 of the terminal device 300 also functions as the notification section 320, the notification unit 240 may perform processing of causing the display to display first image data in Step S204 and perform processing of causing the display to display second image data in Step S205. For example, images, icons, texts, and the like included in the first image data are different from those included in the second image data.

As has been described above, the notification unit 240 of this embodiment may determine the mode of notification of the notification processing based on the patient load information. For example, as described previously, the notification unit 240 may use different display modes between where the value of the patient load information is the predetermined threshold or more and the case where the value of the patient load information is below the predetermined threshold. In addition, the number of display modes is not limited to two, and three or more display modes may be used. Although details thereof will be described later using FIG. 8 and the like, the value of the patient load information tends to increase when the state of the patient is bad, for example. Accordingly, the method of this embodiment makes it possible to show, at the time of notification processing, loads needed to deal with the notification processing and the degree of priority (the degree of urgency) of the notification processing to a caregiver. As a result, this can prompt the caregiver to do an appropriate action. For example, the caregiver can do actions such as determining the priorities appropriately when multiple pieces of notification processing occur at the same time and asking another caregiver to become in charge instead in an early phase when the caregiver finds it difficult to deal with the notification processing.

Note that, if the notification conditions are not met (Step S202: No), the processing of Steps S203 to S205 will be omitted.

### 3.2 Hospital Ward Load Information

Next, hospital ward load information used for notification setting processing will be described in detail. The notification setting unit 210 of this embodiment may obtain patient load information. For example, the notification setting unit 210 obtains the patient load information in the processing of obtaining the hospital ward load information illustrated in Step S101 of FIG. 5. Specifically, a patient in this embodiment includes multiple patients who are hospitalized in a hospital ward to be subjected to processing, and the notification setting unit 210 may obtain, for each of the multiple patients, the patient load information indicating the degree of loads needed for a caregiver to deal with the notification processing on per-patient basis when notification processing is performed. Then, based on the patient load information corresponding to the multiple patients, the notification setting unit 210 obtains the hospital ward load information indicating the degree of loads in the hospital ward. According to the method of this embodiment, it is possible to presume a load needed to deal with each of the patients by obtaining the patient load information. For example, the notification setting unit 210 can obtain the patient load information in consideration of a difference among the patients. Accordingly, by obtaining the hospital ward load information based on the patient load information of the multiple patients, it is possible to increase the accuracy of the hospital ward load information. Hereinbelow, a flow of processing will be described in detail.

FIG. 7 is a flowchart illustrating processing of obtaining the hospital ward load information illustrated in Step S101 of FIG. 5. The processing illustrated in FIG. 7 may be executed for each bed 100 provided in a hospital, for example.

In Step S301, the notification setting unit 210 determines whether the bed 100 to be subjected to processing is used by a patient. For example, the memory 250 of the information processor 200 may store, as management data, data associating a bed ID of the bed 100 with a patient ID of the patient using this bed 100. The notification setting unit 210 may read the data from the memory 250 and determine whether the patient ID is associated with the bed 100 to be subjected to processing.

If determining that the patient is on the bed 100 (Step S301: Yes), in Step S302, the notification setting unit 210 sets a value of the patient load information of the target patient to 1. Note that, 1 mentioned here is an example of a reference value as the value of the patient load information, and other values may be set as this value.

Next, in Step S303, the notification setting unit 210 adds a predetermined value to the value of the patient load information based on load master data. For example, the notification setting unit 210 may obtain the patient load information based on a risk of falling down/falling off including at least one of the patient's risk of falling down and the patient's risk of falling off. The risk of falling down indicates the patient's risk of falling down. For example, falling down mentioned here indicates falling down at various locations and scenes such as falling down during walking, falling down in a toilet, and falling down during ascending/descending stairs. Meanwhile, the risk of falling off indicates the patient's risk of falling off the bed 100, a chair, a wheelchair, or the like. This makes it possible to set the patient load information according to the level of the risk of falling down/falling off. For example, the memory 250 may store information, indicating the patient's risk of falling down/falling off, in association with this patient, and the notification setting unit 210 may read the information from the memory 250.

FIG. 8 illustrates an example of load master data in this embodiment. As illustrated in No. 1 to 3 of FIG. 8, for example, the load master data of this embodiment may include data associating the degree of the risk of falling down/falling off with an additional value of the patient load information. Here, the degree of the risk of falling down/falling off is evaluated in three levels of I, II, and III, and values of 0.5, 1.0, and 1.5 are associated with these levels respectively as the corresponding additional values (load values). In the risk of falling down/falling off, I indicates a relatively low-risk state and III indicates a relatively high-risk state. Note that, although not illustrated in FIG. 8, a level indicating that the risk of falling down/falling off is sufficiently low (such as no risk of falling down/falling off) may be provided, and an additional value of 0 may be associated with this level.

For example, in Step S303, the notification setting unit 210 identifies the level of the risk of falling down/falling off by referring to an electronic medical record of the patient to be subjected to processing. Further, the notification setting unit 210 identifies the additional value according to the patient's risk of falling down/falling off by referring to the load master data of FIG. 8 and adds the additional value thus identified to the value of the patient load information. For example, in a case where the risk of falling down/falling off of the patient whose value of the patient load information is set to 1 by the processing of Step S302 is III, the notification setting unit 210 updates the value of the patient load information to 2.5 by adding 1.5 to the patient load information.

Alternatively, the notification setting unit 210 may obtain the patient load information based on information on a room of the patient. The information mentioned here is the distance from a nurse station which is a base of a caregiver, for example. As illustrated in No. 4 and 5 of FIG. 8, for example, the load master data of this embodiment may include data associating the distance from the nurse station to the room of the patient with the additional value of the patient load information. Here, the distance is evaluated in two levels of near and far, and values of 0.0 and 1.5 are associated with these levels respectively as the corresponding additional values (load values). This makes it possible to set the patient load information according to accessibility to the room of the patient.

FIG. 9 illustrates an example of hospital room master data indicating the attribute of a hospital room. The hospital room master data is information associating a hospital ward code for identifying a hospital ward, a hospital room code for identifying a hospital room, and the distance from a target hospital room to the nearest nurse station with each other. The hospital room master data is stored in the memory 250, for example. In addition, the memory 250 may store, as management data, data associating a patient ID of a patient with a room ID (hospital room code) indicating the room of the patient.

In Step S303, the notification setting unit 210 reads the data from the memory 250 to identify the hospital room code indicating the room of the patient. The notification setting unit 210 identifies the distance from the room of the patient to the nurse station by referring to the hospital room master data. Further, the notification setting unit 210 identifies the additional value according to the distance by referring to the load master data of FIG. 8 and adds the additional value thus identified to the value of the patient load information. For example, in a case where the distance between the nurse station and the room of the patient whose value of the patient load information is set to 1 by the processing of Step S302 is “far”, the notification setting unit 210 updates the value of the patient load information to 2.5 by adding 1.5 to the patient load information.

Note that, although the distance with the nurse station set as a reference has been exemplified above, the method of this embodiment is not limited to this. For example, in a case where position information of a caregiver can be measured, the notification setting unit 210 may periodically obtain the current position of a caregiver who is in charge of a patient and the position of the room of the patient and update the patient load information based on the distance therebetween and the load master data.

In addition, the hospital room master data may include information indicating an attribute other than the distance of a hospital room. The attribute of the hospital room is information indicating whether the hospital room is a normal hospital room or a clean room, for example. In this case, the load master data of FIG. 8 may include data associating the normal hospital room with an additional value of 0.0 and data associating the clean room with an additional value of 2.0. In this example, in a case where the clean room is set as a target, due to a heavy load because of the need to do sterilization and wear a clean suit, the additional value corresponding to the clean room is relatively large.

Alternatively, the notification setting unit 210 may obtain patient load information based on disease state information on the disease state of a patient. For example, although not illustrated in FIG. 8, the load master data of this embodiment may include data associating the disease state of the patient with the additional value of the patient load information. For example, the load master data may include data associating a disease state “limb disorder” with an additional value of 1.0 and data associating a disease state “brain infarction” with an additional value of 2.0. The load master data may also include data associating a disease state “within Y days after operation” with an additional value of Z. Here, each of the values Y and Z may be configured so that only one value is settable, or sets of multiple values are settable. For example, an additional value of 3.0 may be set for “within three days after operation”, and an additional value of 1.5 may be set for “within seven days after operation”. In addition, the disease state (disorder, past medical history) included in the load master data is not limited to the above example, and various disease states such as a vision disorder, dementia, and epilepsia may be employed. The disease state information is stored in the memory 250, for example.

In this event, the notification setting unit 210 may obtain the patient load information based on an electronic medical record of the patient. The electronic medical record includes information indicating a result of diagnosis by a doctor, for example. The information processor 200 of this embodiment is assumed to be able to link up with the system in the hospital as described previously, and the electronic medical record is stored in the memory 250, for example. Accordingly, since a processing load at the time of referring to the electronic medical record is assumed to be low, it is possible to obtain the patient load information using accurate information based on professional's judgment without increasing a system load.

Further, the processing of obtaining the patient load information is not limited to processing using only one of the risk of falling down/falling off, the hospital room, and the disease state, and may be processing using a combination of two or more of them. For example, in a case where the level of the patient's risk of falling down/falling off is II and the hospital room is away from the nurse station, the patient load information with a reference value of 1 may be updated with 3.5 obtained by adding 1.0 and 1.5 to the reference value.

The patient load information of the target patient is obtained by the processing of Steps S302 and S303 described above. Next, in Step S304, the notification setting unit 210 determines whether notification setting for the target patient is turned on (some sort of notification condition has already been associated with the target patient). If the notification setting is turned on, the value of the patient load information is added to the hospital ward load information. On the other hand, if the notification setting is turned off, since no notification processing for the target patient is executed, no load is assumed to be generated in dealing with the notification processing for this patient. In this case, the value of the patient load information is not added to the hospital ward load information. By executing the processing of Step S304 for all the patients hospitalized in the target hospital ward, a sum of the patient load information of the patients whose notification setting has been made is obtained as the hospital ward load information. As has been described above, since the patient load information is information reflecting each patient's state, the hospital ward load information is considered as a value obtained by precisely presuming loads for a caregiver to deal with the notification processing on per-hospital ward basis.

### 3.3 Recommended Condition for Executing Notification Processing

Further, after calculating patient load information and updating hospital ward load information as needed (Steps S302 to S304 of FIG. 7), the notification setting unit 210 may obtain a notification condition recommended for a target patient. For example, in Step S305, the notification setting unit 210 determines whether the patient's state coincides with any of determination master data. Hereinbelow, some of specific examples of the determination master data will be described.

### 3.3.1 Allowable Moving Range

For example, the notification setting unit 210 may obtain a condition for executing notification processing recommended for a patient based on the patient's allowable moving range. FIG. 10 is an example of determination master data used for processing of determining a recommended notification condition. As illustrated in FIG. 10, the determination master data mentioned here may be data associating an allowable moving range of the patient instructed by an electronic medical record, a pictogram, and the recommended notification condition with each other.

For example, if a caregiver can comprehend information such as an electronic medical record in detail, the caregiver can provide care appropriate for each patient. However, since a caregiver is assumed to be in charge of multiple patients, it is not easy for the caregiver to precisely comprehend a lot of information on all the patients. Further, some caregivers are in charge of care for a patient supplementarily, and some caregivers are in charge of only a specific field such as rehabilitation. Such caregivers have few chances of contact with a target patient, and thus it may be difficult for them to comprehend detailed information on this patient.

To deal with this, in this embodiment, patient's states may be managed by associating them with pictograms which are visually easy to understand. In the example of FIG. 10, the patient's state indicates an allowable moving range, and specifically includes states such as “free inside hospital”, “free inside hospital ward”, “free inside hospital room”, and “movement prohibited”. The term “free inside hospital” indicates a state where movement inside a hospital is not restricted. The term “free inside hospital ward” indicates a state where movement inside a hospital ward is not restricted but movement outside the hospital ward is restricted. The term “free inside hospital room” indicates a state where movement inside a hospital room is not restricted but movement outside the hospital room is restricted. The term “movement prohibited” indicates a state where movement away from the bed 100 is restricted.

For example, in this embodiment, the states “free inside hospital”, “free inside hospital ward”, “free inside hospital room”, and “movement prohibited” may be respectively assigned with pictogram codes that do not overlap with each other. Then, one pictogram code is associated with one pictogram. The pictogram codes mentioned here are numeric data, for example, but data of another format may be used. In the example of FIG. 10, the four pictogram codes corresponding to the states “free inside hospital”, “free inside hospital ward”, “free inside hospital room”, and “movement prohibited” are respectively associated with humanoid pictograms with different colors and patterns.

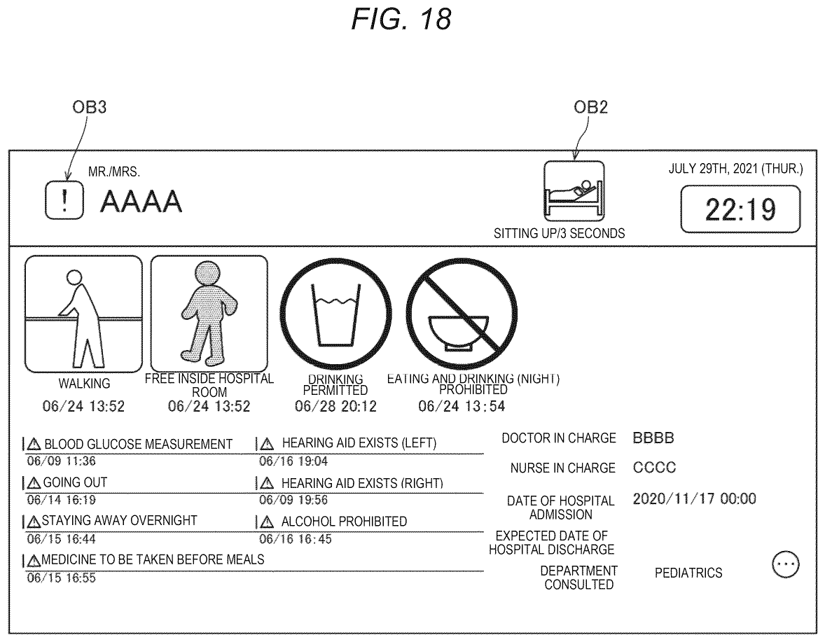

By associating the patient's states with the pictograms in this manner, it is possible to inform a caregiver of the patient's states using the pictograms which are visually easy to understand. By using the pictograms illustrated in FIG. 10 for example, even a caregiver having few chances of contact with a patient can easily comprehend an allowable moving range of the patient. An example of display screen using pictograms will be described later using FIG. 18. However, in this embodiment, the pictogram codes do not necessarily have to be used, and other data may be used as long as patient's states such as the patient's allowable moving range are identifiable.

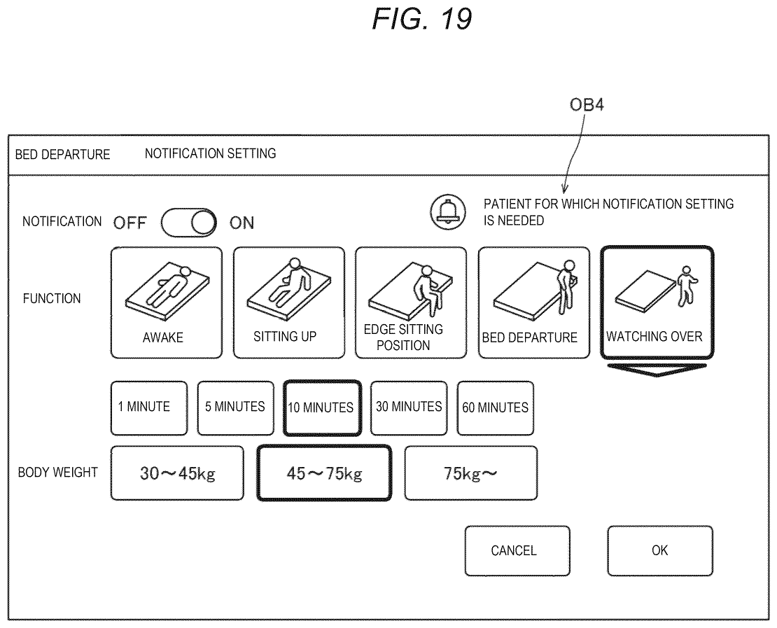

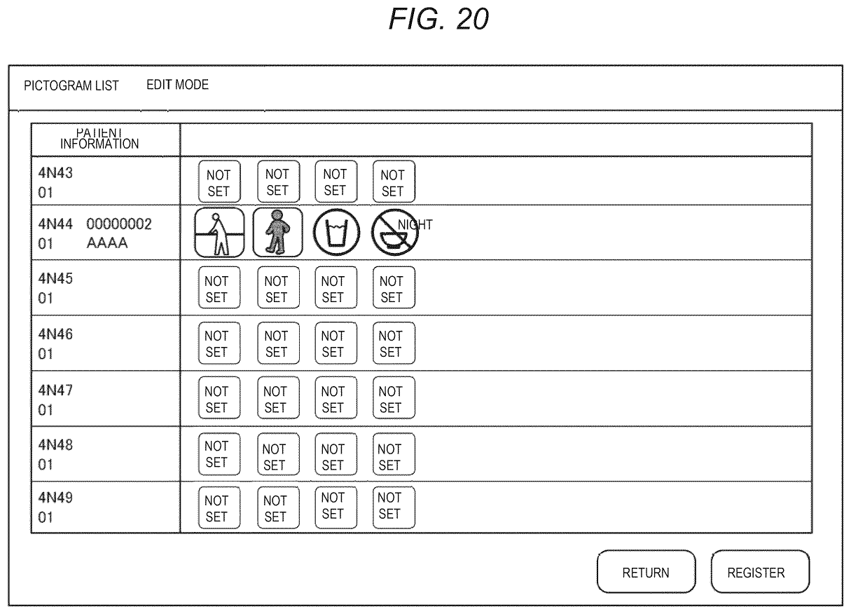

For example, the notification setting unit 210 acquires a pictogram code assigned to a patient by reading an electronic medical record from the memory 250. Note that, processing of assigning a pictogram code (pictogram) to a patient will be described later using FIGS. 19 and 20.

Then, in Step S305 of FIG. 7, the notification setting unit 210 determines, based on the determination master data of FIG. 10, whether a pictogram code matching any of the states “free inside hospital”, “free inside hospital ward”, “free inside hospital room”, and “movement prohibited” exists in the pictogram code assigned to the patient. If the pictogram code assigned to the patient corresponds to the determination master data (Step S305: Yes), in Step S306, the notification setting unit 210 determines a recommended notification condition based on the determination result using the determination master data.

For example, if the pictogram code corresponding to the state “free inside hospital” is assigned to the patient, the notification setting unit 210 determines that no recommended notification condition is needed (no recommended notification condition exists) based on the determination master data of FIG. 10. If the pictogram code corresponding to the state “free inside hospital ward” is assigned to the patient, the notification setting unit 210 determines that a recommended notification condition is “watching over”. If the pictogram code corresponding to the state “free inside hospital room” is assigned to the patient, the notification setting unit 210 determines that a recommended notification condition is “bed departure”. If the pictogram code corresponding to the state “movement prohibited” is assigned to the patient, the notification setting unit 210 determines that a recommended notification condition is “edge sitting position” or “sitting up”.

This makes it possible to prompt a user to set an appropriate notification condition according to an allowable moving range. For example, in the display processing illustrated in Step S104 of FIG. 5, the display processor 220 may display a setting screen, including a recommended notification condition, on the display. A specific example thereof will be described later using FIG. 17. Note that, if the pictogram code assigned to the patient does not correspond to the determination master data (Step S305: No), the processing of Step S306 is omitted.

### 3.3.2 Moving Means

Meanwhile, the processing using the determination master data illustrated in Step S305 is not limited to the above example. For example, the notification setting unit 210 may obtain a condition for executing notification processing recommended for a patient based on the patient's moving means.

FIG. 11 illustrates another example of determination master data. The determination master data illustrated in FIG. 11 may include, for example, pictogram codes indicating moving means of a patient, types of notification condition (determination data), determination conditions, condition determination expressions, and attributes.

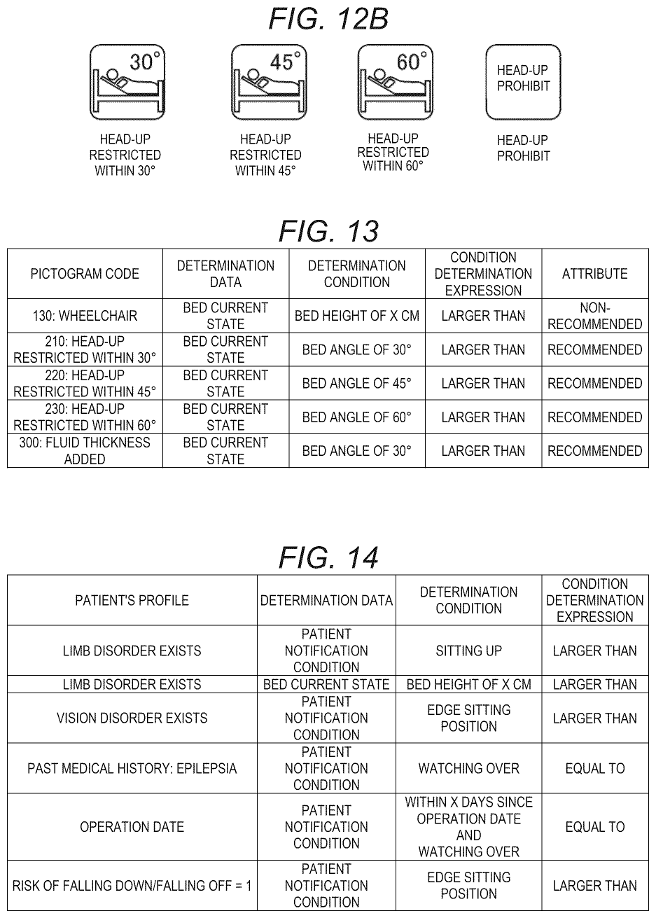

As in the above example, the pictogram codes are each associated with a specific pictogram. FIG. 12A illustrates pictograms associated with the moving means of a patient. For example, in this embodiment, divisions such as “walking”, “stick”, “wheelchair”, “wheeled walker”, and “movement in bed” are set as the moving means and are respectively assigned with pictogram codes. Here, the pictogram code for “walking” is 110, the pictogram code for “stick” is 120, and the pictogram code for “wheelchair” is 130. Likewise, the divisions “wheeled walker” and “movement in bed” are also assigned with pictogram codes not overlapping with the codes indicating other moving means. Among the pictograms illustrated in FIG. 12A, the pictogram indicating “walking” is associated with the pictogram code 110, the pictogram indicating “stick” is associated with the pictogram code 120, and the pictogram indicating “wheelchair” is associated with the pictogram code 130.

Note that, as illustrated in FIG. 12A, data including texts and the like may be used for states difficult to represent in pictograms, such as “rest in bed”. In this embodiment, for example, reminder codes may be assigned to the states difficult to represent in pictograms, and each reminder code may be associated with data including texts indicating this state. In this case, the patient's state can also be presented as a text icon, thus making it possible to inform a caregiver of the patient's state in an easy-to-understand manner.

Referring back to FIG. 11, the description is continued. In the determination master data illustrated in FIG. 11, determination data, a determination condition, and a condition determination expression are associated with each pictogram code. The determination data indicates the attribute of a notification condition, and indicates here that the data is intended for patient notification conditions (notification conditions on the position, posture, sleeping/awake of a patient in a narrow sense). The determination condition indicates criteria of conditions determined based on the patient's state. The condition determination expression is information indicating the range of conditions, and specifically, may be data indicating any of “equal to”, “larger than”, and “smaller than” the determination condition.

Note that, the condition determination expression “larger than” indicates a unit of measurement in which the patient's activity is allowed, for example, and the magnitude relationship may be defined for the patient notification condition in the order of (“watching over”>“bed departure”>“edge sitting position”, >“sitting up”>“awake”), for example. Here, in a case where there is a notification condition “not set” indicating that no notification condition has been set, this condition “not set” may be set to be larger than the condition “watching over”, or may be set to be smaller than the condition “awake”. Further, as will be described later using FIG. 13, in a case where the notification condition is a condition on the movable range of the bed 100 (bed current state), the magnitude relationship may be determined based on numeric values of heights and angles.

The condition is identified by a combination of the determination condition and the condition determination expression. The attribute in FIG. 11 is data for identifying whether the identified condition is a recommended notification condition or a non-recommended notification condition. Note that, FIG. 11 illustrates an example in which a column for identifying recommended/non-recommended is included in the determination master data, but processing of this embodiment is not limited to this. For example, a list consisting of data including recommended conditions (white list) and a list consisting of data including non-recommended conditions (error list) may be respectively managed as different sets of determination master data.

The notification setting unit 210 identifies a pictogram code associated with a patient, and identifies a recommended notification condition or a non-recommended notification condition based on the determination condition and the condition determination expression associated with this pictogram code. Then, if the notification condition having been set for the patient does not coincide with the recommended notification condition or if the notification condition having been set for the patient coincides with the non-recommended notification condition, the notification setting unit 210 prompts a change of the set notification condition. Further, if no notification condition is set for the patient, the notification setting unit 210 may prompt an addition of a notification condition. The change/addition of the notification condition is performed by displaying a setting screen on the display (the display 260 or the display 310) via the display processor 220, for example.

A specific example will be described. For example, assume a case where a target patient is assigned with data indicating “walking” (110) as the pictogram code. In this case, first, the notification setting unit 210 searches for the pictogram code that corresponds to “walking” from the determination master data of FIG. 11, and thereby extracts data on the first row (Step S305 of FIG. 7). Then, since the determination condition is “watching over”, the condition determination expression is “equal to”, and the attribute is “recommended”, the notification setting unit 210 determines that the recommended notification condition is “watching over” (Step S306). This is because, if the pictogram code is “walking”, the target patient is considered to be able to walk on his/her own, and thus there seems to be low necessity of finely managing his/her movement on the bed 100 and bed departure for a short period of time.

Next, the notification setting unit 210 compares the notification condition having been set for the target patient with the recommended notification condition. Here, assume a case where the notification condition “edge sitting position” has been set for the target patient. In this case, since the conditions “edge sitting position” and “watching over” are different, the notification setting unit 210 determines that the set notification condition does not coincide with the recommended condition. Accordingly, the notification setting unit 210 outputs “watching over” as the recommended notification condition. For example, in Step S104 of FIG. 5, the display processor 220 may display a setting screen, including the recommended notification condition, on the display.

The same goes for a case of using other pictogram codes. For example, in a case where the pictogram code is “stick”, the target patient needs a stick but is able to move on his/her own to some degree, and thus there is low necessity of finely managing his/her movement on the bed 100 and bed departure for r a short period of time. Accordingly, the notification setting unit 210 determines to set the condition “watching over” as the recommended notification condition based on the determination condition, the condition determination expression, and the attribute. Then, the notification setting unit 210 determines whether to perform processing of presenting the recommended notification condition to a user based on processing of comparison between the set notification condition and the recommended notification condition.

Meanwhile, in a case where the pictogram code is “wheelchair”, the determination condition is “edge sitting position”, the condition determination expression is “larger than”, and the attribute is “non-recommended”. Accordingly, the notification setting unit 210 determines to set the conditions “watching over” and “bed departure” as the non-recommended notification conditions based on the magnitude relationship (“watching over”>“bed departure”>“edge sitting position”, >“sitting up”> “awake”). Since a patient using a wheelchair cannot move on his/her own, a caregiver needs to provide assistance in movement before the patient departs from the bed such as when the patient takes an edge sitting position. In other words, the data mentioned here corresponds to a case where the conditions “watching over” and “bed departure” in which notification processing is executed at the time of bed departure or after bed departure are deemed not preferable.

Then, the notification setting unit 210 performs processing of comparing the set notification condition with the non-recommended notification condition. For example, if the set notification condition is included in the non-recommended notification condition, the notification setting unit 210 may perform processing of identifying notification conditions other than the non-recommended notification condition as the recommended notification condition (Step S306 of FIG. 7). Here, the notification conditions other than the non-recommended notification condition are patient notification conditions excluding the conditions “watching over” and “bed departure”, and are a set of the conditions “edge sitting position”, “sitting up”, and “awake”, for example.

Meanwhile, the fourth row of FIG. 11 is an example indicating a state where the pictogram code is “notification setting needed”. For example, a patient having some sort of problem with a disease state or the like is assigned with the pictogram code “notification setting needed”. In this case, the determination condition is “not specified”, the condition determination expression is “equal to”, and the attribute is “non-recommended” The condition “not specified” indicates a null set in which none of the conditions “watching over”, “bed departure”, “edge sitting position”, “sitting up”, and “awake” belonging to the patient notification condition is set. In other words, in this case, the notification condition which is a null set is determined as the non-recommended notification condition. For example, if none of the notification conditions included in the patient notification condition is set, since the current notification condition coincides with the non-recommended notification condition, the notification setting unit 210 recommends a change of the notification condition. The recommended notification condition is a complementary set of the null set, and is thus a set of the conditions “watching over”, “bed departure”, “edge sitting position”, “sitting up”, and “awake” belonging to the patient notification condition. In other words, the notification setting unit 210 recommends setting of an arbitrary patient notification condition.

Note that, the above shows the example in which the data attributes on the first and second rows of FIG. 11 are “recommended” and the data attributes on the third and fourth rows thereof are “non-recommended”, but are not limited to this.

For example, when we consider a patient using a wheelchair, it is possible to notify a caregiver in advance of the fact that assistance in movement needs to be provided for the notification conditions “edge sitting position”, “sitting up”, and “awake”, but a burden on the caregiver might increase when notification processing occurs frequently. To deal with this, from the perspective of suppressing the notification frequency and focusing on detection of an anomaly where a wheelchair user moves away from the bed 100, the possibility of setting the notification conditions “watching over” and “bed departure” on purpose is also conceivable.

In this case, the data attribute on the third row of the determination master data of FIG. 11 is “recommended”. In this example, the attribute varies depending on whether courteous care is prioritized or a reduction of the notification frequency is prioritized. As can be understood from the above example, in this embodiment, data including the same determination condition and the condition determination expression may be treated as data indicating the recommended notification condition or may be treated as data indicating the non-recommended notification condition. For example, a value in the column of the attribute illustrated in FIG. 11 may be changed based on the user's operation. Alternatively, the notification setting unit 210 may change the value of the attribute based on the relationship between the latest hospital ward load information and the allowable load information.

In addition, the fact that an attribute can be changed is not limited to data corresponding to “wheelchair” but also data on other rows of the determination master data illustrated in FIG. 11 and data on each row of determination master data which will be described later using FIG. 13 and FIG. 14.

### 3.3.3 Bed Movable Range

Meanwhile, the pictogram codes used in this embodiment and the pictograms associated with these pictogram codes are not limited to the moving means. For example, as illustrated in FIG. 12B, the patient's state may indicate the movable range of the bed 100 recommended for a patient. For example, in this embodiment, divisions such as “30° or smaller”, “45° or smaller”, and “60° or smaller” are set in advance as the back angle of the bed 100 and pictogram codes are assigned to them respectively. For example, the pictogram code of “30° or smaller” is 210, the pictogram code of “45° or smaller” is 220, and the pictogram code of “60° or smaller” is 230. Among the pictograms illustrated in FIG. 12B, the pictogram indicating “30° or smaller” is associated with the pictogram code 210, the pictogram indicating “45° or smaller” is associated with the pictogram code 220, and the pictogram indicating “60° or smaller” is associated with the pictogram code 230. In addition, a reminder code and data including texts may be used for a state “head-up prohibited”.

FIG. 13 illustrates another example of determination master data. As in the determination master data illustrated in FIG. 11, the determination master data illustrated in FIG. 13 may include data associating pictogram codes, types of notification condition (determination data), determination conditions, condition determination expressions, and attributes with one another. The determination data mentioned here is a bed current state indicating the notification condition on the movable range of the bed 100.

For example, assume a case where a target patient is assigned with 130, which corresponds to “wheelchair”, as the pictogram code. In this case, in Step S305 of FIG. 7, the notification setting unit 210 searches for the pictogram code that corresponds to “wheelchair” from the determination master data of FIG. 13, and thereby extracts data on the first row. Then, since the determination condition is “bed height of X cm”, the condition determination expression is “larger than”, and the attribute is “non-recommended”, in Step S306 of FIG. 7, the notification setting unit 210 determines that the condition “larger than bed height X cm” is the non-recommended notification condition. Accordingly, the notification setting unit 210 determines that the recommended notification condition is “bed height X cm or smaller”. When a patient using a wheelchair is set as a target, the patient needs to move from the bed 100 to the wheelchair. Further, the fact that it is easier to provide assistance in the movement with the bed 100 having a relatively high height has been known. In other words, since the condition “larger than bed height X cm” is a preferable state and no notification is needed for this state, by setting the reverse notification condition “bed height X cm or smaller”, it is possible to inform a caregiver of the fact that the height of the bed 100 is low and not suitable for the movement.

The notification setting unit 210 determines whether the set notification condition coincides with the recommended notification condition “bed height X cm or smaller”. If these conditions do not coincide with each other, the notification setting unit 210 performs processing of presenting the recommended notification condition “bed height X cm or smaller” to a user.

Meanwhile, if the pictogram code indicating “head-up restricted within 30°” is assigned to a patient, the notification setting unit 210 extracts data on the second row of FIG. 13. The notification setting unit 210 determines that the recommended notification condition is “larger than bed angle 30°” based on the determination condition, the condition determination expression, and the attribute. The notification setting unit 210 determines whether the set notification condition coincides with the recommended notification condition “larger than bed angle 30°”. If these conditions do not coincide with each other, the notification setting unit 210 performs processing of presenting the recommended notification condition to a user. This makes it possible to recommend notification setting of warning the excessively high back angle to a user whose excessive head-up is not preferable. The same goes for “head-up restricted within 45°” and “head-up restricted within 60°”.

Meanwhile, if the pictogram code indicating “fluid thickness” is assigned to a patient, the notification setting unit 210 extracts data on the fifth row of FIG. 13. The code “fluid thickness” is a pictogram code indicating that food and drink of a target patient are thickened to a certain degree. The notification setting unit 210 determines that the recommended notification condition is “smaller than bed angle 30°” based on the determination condition, the condition determination expression, and the attribute. A patient for whom food and drink need to be thickened has a low swallowing ability, so it is preferable to increase the back angle of the bed 100 to a certain degree during eating and drinking. In other words, if such a patient is set as a target, by executing notification processing if the bed angle is a predetermined degree or smaller, it is possible to prompt adjustment of the back angle for facilitating swallowing.

The notification setting unit 210 determines whether the set notification condition coincides with the recommended notification condition “smaller than bed angle 30°”. If these conditions do not coincide with each other, the notification setting unit 210 performs processing of presenting the recommended notification condition to a user. This makes it possible to recommend assistance in swallowing by moving upward a head part of a patient having a low swallowing ability.

### 3.3.4 Combination of Disease State, etc.

Using FIGS. 10 to 13, the description has been given of the processing based on the information associated with the pictogram codes such as the allowable moving range, the moving means, and the movable range of the bed 100. However, the method of this embodiment is not limited to the processing using the pictogram codes.

FIG. 14 illustrates another example of determination master data. The determination master data illustrated in FIG. 14 may include, for example, patient's profiles, types of notification condition (determination data), determination conditions, and condition determination expressions. In addition, although not illustrated in FIG. 14, the determination master data may include attributes for identifying recommended/non-recommended as in the examples of FIG. 11 and FIG. 13.

As illustrated in FIG. 14, the patient's profile may be a disease state such as a disorder, a past medical history, an operation date, and a risk of falling down/falling off. The determination data, the determination condition, and the condition determination expression are the same as those of the determination master data of FIG. 11 and the like. This makes it possible to recommend an appropriate notification condition according to the patient's state (disease state) even in the case of disease states which are difficult to describe using pictogram codes.

For example, for a patient having a limb disorder, a notification condition in consideration of detection of sitting up and the bed height with which the patient is easy to move may be recommended. Meanwhile, for a patient having a vision disorder, a notification condition based on the condition “edge sitting position” may be recommended in consideration of the fact that the patient needs to be assisted when moving away from the bed 100. Meanwhile, for a patient having epilepsia in his/her past medical history, a notification condition based on the condition “watching over” may be recommended because unexpected falling down and the like is likely to occur. For a patient having an operation history, a notification condition according to the number of days elapsed since an operation date and a notification condition based on the condition “watching over” may be recommended. Meanwhile, for a patient having a slight degree of risk of falling down/falling off (a patient whose level of the risk of falling down/falling off is I), a notification condition based on the condition “edge sitting position” may be recommended from the perspective of suppressing the patient's falling off the bed 100 and falling down during movement.

Besides, disease states included in the patient's profile and conditions associated with these disease states are not limited to those illustrated in FIG. 14 and various modifications can be made.

Meanwhile, although the various kinds of determination master data have been exemplified above, it is also conceivable that the state of the patient or the bed 100 coincides with multiple records included in the determination master data. Each record mentioned here indicates data of one row in the determination master data. To coincide with the record indicates a case where the patient's state or the like meets the condition determination expression included in the record.

For example, a case where the patient's state meets the condition determination expression of the first record whose attribute is “recommended” and meets the condition determination expression of the second record whose attribute is “non-recommended” is conceivable. In this case, the notification condition to be recommended will be set if the first record is taken into consideration, so that recommendation of a change is not needed. On the other hand, the notification condition to be non-recommended will be set when the second record is taken into consideration, so that recommendation of a change is preferable. In this case, the notification setting unit 210 may perform processing on all the records whose condition determination expressions are met. For example, the notification setting unit 210 may extract the records whose attribute is “recommended” but whose condition determination expressions are not met and all the records whose attribute is “non-recommended”but whose condition determination expressions are met, and may recommend the recommended notification conditions for all these records.

Alternatively, the notification setting unit 210 may set priorities for the records and execute processing on only the records with a relatively high priority. For example, the method of this embodiment may use determination master data in which conditions indicated by multiple records conflict with one another. In this case, priorities may be set at least for the records whose conditions conflict with one another and processing may be executed according to the priorities.

Still alternatively, the method of this embodiment may use determination master data having both records whose notification conditions are similar and easy to meet (loose conditions) and records whose notification conditions are not easy to meet (strict conditions). For example, in the case of using the pictogram codes as illustrated in FIG. 11, the determination master data may include multiple records for the same pictogram code. For example, a record indicating the recommendation condition “watching over” and “equal to” and a record indicating the recommendation condition “bed departure” and “equal to” may coexist for the pictogram code “walking”. In this case, since the latter case performs notification in response to “bed departure” which is a previous phase of “watching over”, notification processing is more likely to occur. In this case, as in the above example, priorities may be set for the records and processing may be executed according to the priorities. In this case, the records with loose conditions may be prioritized, or the records with strict conditions may be prioritized.

### 3.4 Allowable Load Information

Next, a description will be given of processing of obtaining allowable load information illustrated in Step S102 of FIG. 5. The notification setting unit 210 may determine allowable load information based on a history of hospital ward load information and a history of time information indicating the time needed for a caregiver to deal with notification processing. This makes it possible to obtain, as allowable load information, the degree of loads that a caregiver is presumed to be capable of handling based on the past care history.

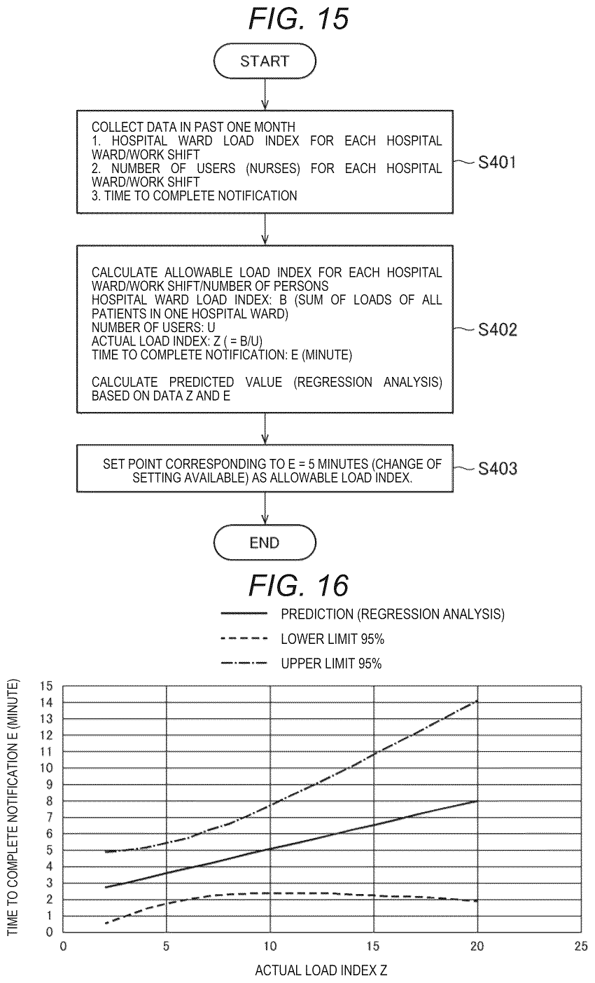

FIG. 15 is a flowchart illustrating processing of obtaining allowable load information. First, in Step S401, the notification setting unit 210 reads past data from the memory 250. For example, the notification setting unit 210 acquires a history of hospital ward load information in the past one month, a history of the number of caregivers working at a target hospital ward, and a history of the time needed for a caregiver to deal with notification processing since the notification processing is made to the caregiver.

As has been described above using FIG. 7, since the notification setting unit 210 can obtain patient load information from information such as an electronic medical record and obtain hospital ward load information, the notification setting unit 210 can acquire a history of the hospital ward load information by managing the hospital ward load information, obtained at each timing, in association with this timing. In addition, since when and which hospital ward caregivers have been on duty are identifiable based on a hospital work record, the notification setting unit 210 can acquire a history of the number of caregivers by referring to this work record.

In addition, the information processor 200 may store, in the memory 250, a history of what kind of notification processing has been performed on which patient. Further, when a caregiver has dealt with notification processing, the caregiver may register information indicating the same to the information processor 200 via the terminal device 300. By referring to the information on the notification processing and how the caregiver has dealt with it, the notification setting unit 210 can acquire a history of the time needed for the caregiver to deal with the notification processing since the notification processing has been made.

In Step S402, the notification setting unit 210 obtains an actual load index Z by dividing a value of the hospital ward load information by the number of caregivers. The actual load index Z is information indicating a load on per-caregiver basis in the past history. This enables the notification setting unit 210 to acquire multiple sets of values (Z, E) of the actual load index Z and the time E needed to complete dealing with the notification processing.

Further, in Step S402, the notification setting unit 210 obtains a regression expression Z=f(E) by conducting regression analysis based on the multiple sets of the values (Z, E). Note that, the function f mentioned here may be a linear function or a nonlinear function. Various methods of obtaining a regression expression are known and those methods are widely applicable in the embodiment, and thus a detailed description thereof will be omitted.

In Step S403, the notification setting unit 210 acquires goal information indicating a time goal in dealing with notification processing. The goal information may be a previously set value, or may be numeric data that a user inputs in a setting screen which will be described later using FIG. 17. The notification setting unit 210 obtains f(T) from goal time T indicated by the goal information and the regression expression.

Here, the value of f(T) indicates an actual load index that is presumed to be capable of completing dealing with notification processing within the goal time T based on the past history. In addition, as has been described above, the actual load index is information indicating a load on per-caregiver basis in the past history. Thus, the notification setting unit 210 acquires the number of caregivers working at a target hospital ward during a calculation target period of allowable load information, and obtains the product of the number of caregivers and f(T). This product is a load that the entire hospital ward is expected to be capable of dealing with notification processing within the goal time T, and corresponds to allowable load information in this embodiment.

FIG. 16 is a diagram illustrating the above processing. In FIG. 16, the vertical axis indicates the time E needed to complete dealing with notification processing since the notification processing has been made, and the horizontal axis indicates the actual load index Z. Here, assume a case where a regression expression Z=f(E) is obtained as in a prediction graph illustrated by a solid line in FIG. 16. In this example, assume a case where five minutes is input as the goal time T. In this case, in the prediction graph, the value of the vertical axis is five (minutes) at the point where the value of the horizontal axis is ten. Accordingly, the value of f(T) is ten, which means that a load allowable for one caregiver is ten. Thus, the notification setting unit 210 obtains allowable load information by the product of the number of caregivers who are in charge of a hospital ward and ten which is a load for each person.

Note that, as illustrated in FIG. 16, the notification setting unit 210 may obtain a 95% confidence interval based on the multiple sets of the values (Z, E). In FIG. 16, a chain line is a graph indicating an upper limit 95% and a broken line is a graph indicating a lower limit 95%. The notification setting unit 210 may obtain an actual load index based on goal information (goal time T) and any of the upper limit 95% graph and the lower limit 95% graph, and may obtain allowable load information by the product of the actual load index and the number of caregivers.

In the example of FIG. 16, the upper limit 95% indicates data acquired in a case where it has taken time to deal with notification processing. In other words, in the case of using the upper limit 95%, an allowable load is estimated to be low. This results in prompting a user to make adjustment to decrease the number of patients whose notification conditions are set to on, thus making it possible to reduce a burden on a caregiver.

Meanwhile, the lower limit 95% indicates data acquired in a case where dealing with notification processing has been completed smoothly. In other words, in the case of using the lower limit 95%, an allowable load is estimated to be high. This results in prompting a user to make adjustment to increase the number of patients whose notification conditions are set to on, thus making it possible to provide courteous care for a patient.

Note that, an interval obtained from the past history is not limited to the 95% confidence interval, and various statistics values calculated from the history can be used to determine allowable load information.

In addition, although the description has been given above of the example where the value of the allowable load information is simply the product of f(T) and the number of caregivers, the method of this embodiment is not limited to this. For example, depending on the skill of a caregiver, the caregiver may be able to complete care for a relatively large load within the goal time, or may be only able to execute care for a relatively small load. Accordingly, the notification setting unit 210 may obtain allowable load information by performing processing on the value of f(T) in consideration of the skill of the caregiver.

For example, the notification setting unit 210 may obtain a load allowable for a caregiver by using α×f(T) which is the product of f(T) and a factor α that is determined depending on the skill of the caregiver. For example, the reference sign x indicates a factor whose value is one in the case of a caregiver with a normal skill, becomes larger with a higher skill, and becomes smaller with a lower skill. The skill of the caregiver is determined based on factors, such as qualifications held by the caregiver, the length of his/her service, and assessment by an administrative person, which are stored in the memory 250, for example.

The notification setting unit 210 obtains allowable load information by obtaining the sum of α×f(T) obtained for each caregiver. This enables processing in consideration of the skill of the caregiver, thus making it possible to obtain the allowable load information with high precision.

### 3.5 Display of Screen

Next, a description will be given of an example of a screen that the display processor 220 displays on the display 260 or the display 310.

### 3.5.1 Setting Screen

FIG. 17 illustrates an example of the setting screen displayed in Step S104 of FIG. 5. For example, on the setting screen, a screen including the number of caregivers at a timing to be subjected to processing, the goal time T which is the goal information, information indicating the relationship between the hospital ward load information and the allowable load information, and a notification condition setting situation for each patient is displayed. Note that, the timing to be subjected to processing may be the current time or may be a future timing.

The example of FIG. 17 shows that the number of caregivers who are in charge of a target hospital ward is ten and five minutes is set as the goal time T.

In addition, an object OB1 is displayed as information indicating the relationship between the hospital ward load information and the allowable load information. The object OB1 includes a semicircular pie chart (doughnut graph) and a pointer-shaped object that points at a predetermined position of the pie chart. For example, the position at which the pointer-shaped object points varies depending on the relationship between the hospital ward load information and the allowable load information. For example, the center of the pie chart indicates a state where the value of the hospital ward load information is equal to the value of the allowable load information. With this state set as a reference, the position at which the pointer-shaped object points moves to the left as the value of the hospital ward load information becomes relatively smaller, and the position at which the pointer-shaped object points moves to the right as the value of the hospital ward load information becomes relatively larger. This makes it possible to present the relationship between the hospital ward load information and the allowable load information to a user in a visually easy-to-understand manner.

However, the information indicating the relationship between the hospital ward load information and the allowable load information may be represented by arranging the value of the hospital ward load information and the value of the allowable load information side by side, may be represented by icons which vary depending on the ratio or difference between the value of the hospital ward load information and the value of the allowable load information, or may be represented by using an object of another mode.

In addition, the example of FIG. 17 shows the notification condition setting situation for each of four patients. For example, the notification condition is not set (off) for a patient A and a patient B. Meanwhile, the notification condition “bed departure” has been set for a patient C and a patient D. This makes it possible to let a user comprehend the notification condition for each of the patients who are hospitalized in a target hospital ward.

Further, the display processor 220 may display, on the display, a setting screen including a condition for executing notification processing recommended for a patient (recommended notification condition). In the example of FIG. 17, the display processor 220 displays, as the recommended notification condition, the condition “sitting up” for the patient A and the patient B and the condition “edge sitting position” for the patient D. This makes it possible not only to notify a user of the current notification condition but also to prompt the user to change the notification condition according to the state of the patient.

The processing of obtaining the set recommended notification condition has been described above using FIG. 10 to FIG. 14. Note that, the recommended notification condition does not necessarily have to be displayed in a case where the target patient does not correspond to any data of the determination master data or a case where the target patient corresponds to data of the determination master data but the set notification condition coincides with the recommended notification condition, for example. In the example of FIG. 17, no recommended notification condition is displayed for the patient C.

In addition, as illustrated in FIG. 17, the display processor 220 may display, together with the recommended notification condition, the reason why this recommended notification condition is recommended. The reason mentioned here may be the pictogram codes in the determination master data of FIG. 11 and FIG. 13 or may be the patient's profiles in the determination master data of FIG. 14, for example. In the example of FIG. 17, the reason “limb disorder” which is the reason of the patient A and the reason “within three days after operation” which is the reason of the patient B correspond to the patient's profiles. The reason “wheelchair” which is the reason of the patient D is the moving means of the patient and corresponds to the pictogram code. This makes it possible to let a user understand the reason of recommendation, and thus possible to support the user in determining whether to employ the recommended notification condition.

In addition, the setting screen may be capable of accepting an operation of changing a notification condition. For example, in FIG. 17, the recommended notification conditions “sitting up” and “edge sitting position” are objects (such as buttons) that can accept a selection operation, and when a user performs an operation of selecting the objects, the operation contents may be transmitted to the notification setting unit 210 of the information processor 200. The notification setting unit 210 performs processing of changing the notification condition of a target patient to the recommended notification condition based on the operation contents thus accepted. In addition, the operation of changing the notification condition is not limited to the selection operation of the recommended notification condition, and a change to any notification condition can be made by a selection operation using a pull-down menu, an input operation using a text box, or the like.

When a notification condition is changed (newly added, for example) and when notification processing on a target patient is performed, a caregiver needs to deal with this. Accordingly, the notification setting unit 210 adds patient load information of the target patient to hospital ward load information to perform processing of updating the hospital ward load information (the processing of returning from Step S106 to S101 of FIG. 5, and Step S304 of FIG. 7). The display processor 220 updates information indicating the relationship between the hospital ward load information and allowable load information by using the updated hospital ward load information. In the example of FIG. 17, the position at which the pointer-shaped object points moves rightward by the update of the hospital ward load information. Note that, the existing notification condition may be deleted, and in this case, the notification setting unit 210 performs processing of updating the hospital ward load information by subtracting the patient load information f the target patient from the hospital ward load information. As a result, in the example of FIG. 17, the position at which the pointer-shaped object points moves leftward.

Meanwhile, the notification setting unit 210 may determine, based on processing of comparison between hospital ward load information and allowable load information, whether to permit setting of a condition for executing notification processing on a patient for whom notification processing has not been set yet. The display processor 220 may display different setting screens between a case where the notification setting unit 210 determines to permit setting of the notification condition and a case where the notification setting unit 210 determines not to permit setting of the notification condition.

For example, as illustrated in FIG. 17, if the value of the hospital ward load information is small relative to the value of the allowable load information and a caregiver still has room for additional care, the notification setting unit 210 may permit addition of a new notification condition. In this case, as illustrated in FIG. 17 for example, the display processor 220 displays the setting screen including the recommended notification conditions of the patient A and the patient B for whom no notification condition has been set yet. On the other hand, if the value of the hospital ward load information is very large relative to the value of the allowable load information and a caregiver has no room for additional care, the notification setting unit 210 may reject addition of a new notification condition. In this case, the display processor 220 displays the setting screen not including the recommended notification conditions of the patient A and the patient B. For example, in FIG. 17, the condition “sitting up” of the patient A and the patient B may be hidden.

Meanwhile, display/non-display of the recommended notification condition do not necessarily have to be determined collectively for multiple patients. For example, in a case where the value of the patient load information of the patient A is small and the value of the patient load information of the patient B is large, addition of a notification condition of the patient A has less influence than that of the patient B. Accordingly, if the value of the hospital ward load information is very large relative to the value of the allowable load information, the display processor 220 may display the setting screen including the recommended notification condition of the patient A but not including the recommended notification condition of the patient B. Alternatively, it is also possible to consider that the patient B whose patient load information is large needs care more than the patient A and thus needs to be prioritized. Accordingly, if the value of the hospital ward load information is very large relative to the value of the allowable load information, the display processor 220 may display the setting screen including the recommended notification condition of the patient B but not including the recommended notification condition of the patient A.

Meanwhile, a decision to display/not to display the recommended notification condition may be made not only in the case of adding a new notification condition but also in the case of changing the notification condition for a patient whose notification condition has been set. For example, as illustrated in FIG. 17, the display processor 220 may determine whether to display the setting screen including the recommended notification condition of the patient D or to display the setting screen not including the recommended notification condition of the patient D, based on the relationship between the hospital ward load information and the allowable load information.

### 3.5.2 Screen Including Pictogram

FIG. 18 illustrates an example of a patient screen on which information on a specific patient is displayed and which includes a pictogram. As illustrated in FIG. 18, the patient screen includes a patient's name, a notification condition, and a pictogram corresponding to a pictogram code.

In FIG. 18, a patient named AAAA is displayed. In addition, as the notification condition, an object OB2 indicating the condition “sitting up” and indicating that a condition of giving a notice after an elapse of three seconds or more since operation detection has been set.

In addition, the display processor 220 may display a screen including an object indicating warning if the notification condition set for the patient and the recommended notification condition are different. In the example of FIG. 18, an object OB3 including ! mark beside the name is displayed. This makes it possible to prompt a user to reconsider the notification condition.

Note that, the example of displaying the object OB3 indicating warning in the screen where the patient's individual information is displayed has been illustrated here, but the present disclosure is not limited to this. For example, the display processor 220 may display a setting screen including an object indicating warning if the execution condition recommended for the patient and the execution condition set for the patient are different. For example, the display processor 220 may display a warning object in the setting screen illustrated in FIG. 17. As has been described above, since the notification condition can be changed in the setting screen, it is useful to prompt reconsideration of the notification condition in this setting screen.

Further, the patient screen may include a pictogram suited to the patient's state. For example, since the moving means of the patient AAAA is “walking”, the pictogram code 110 indicating “walking” is assigned. In this case, the display processor 220 displays the patient screen including the pictogram corresponding to the pictogram code 110. Likewise, in FIG. 18, the patient screen including the pictogram corresponding to the state “free inside hospital room” (FIG. 10), the pictogram corresponding to the state where intake of drink is permitted, and the pictogram corresponding to the state where nighttime eating and drinking is prohibited is displayed.

The patient AAAA is also associated with factors for which special attention needs to be paid, such as factors where the patient needs blood glucose measurement, where the patient wears hearing aids on the right and left ears, where the patient goes out/stays away overnight, where the patient is prohibited from intake of alcohol, and where the patient is prescribed with medicine to be taken before meals. For example, these kinds of information may be managed as reminder codes or may be managed as other information. The patient screen may include texts indicating the contents of the reminder code, for example.

The patient screen may also include information such as a doctor in charge, a nurse in charge, date of hospital admission, expected date of hospital discharge, and a department consulted.

FIG. 19 illustrates an example of a setting screen for setting detailed notification conditions for each patient. The screen illustrated in FIG. 19 may be displayed in a pop-up view when a predetermined operation is executed in the patient screen of FIG. 18, for example. For example, the predetermined operation is an operation of selecting the object OB2 indicating the notification condition, but other operations may be used. The setting of the notification condition in this embodiment may be performed in the screen having been described above using FIG. 17, may be performed in the screen illustrated in FIG. 19, or may be performed in both screens.

In FIG. 19, a toggle button for operating on/off of notification processing (to set or not to set notification condition) is displayed, for example. If the notification processing is set to on by the toggle button, the display processor 220 displays buttons for selecting any of the conditions “awake”, “sitting up”, “edge sitting position”, “bed departure”, and “watching over” as the notification condition related to the patient notification condition. In the example of FIG. 19, since the condition “watching over” is set, the display processor 220 displays multiple buttons for selecting the time to execute notification since movement outside the hospital room (or bed departure). Here, the example of selecting the button of 10 minutes is illustrated. Note that, the load sensors 140 may be used for determination of the patient notification condition. Accordingly, in order to set a load reference value, the display processor 220 may display buttons for prompting input of the body weight of the patient. Once an operation of selecting an “OK” button is performed, the notification setting unit 210 sets the notification condition corresponding to the selected button to the patient. In the example of FIG. 19, since the conditions “watching over” and “10 minutes” are selected, the notification setting unit 210 sets the notification condition “watching over (10 minutes)” to the target patient.

Note that, the example here is one in which no necessary notification setting is made for the patient. Accordingly, as illustrated in FIG. 19, the display processor 220 may display the setting screen including the object OB4 indicating warning. The object OB4 mentioned here is an icon indicating a bell and a warning text; however, as can be understood from the example of the object OB3 in FIG. 18, the mode of the warning object is not limited to this.

FIG. 20 illustrates an edit screen example for associating pictograms (pictogram codes) with a patient. For example, the edit screen may include pictograms associated with each of multiple patients. In the example of FIG. 20, four pictograms such as “walking” and “free inside hospital room” are associated with the patient AAAA (see FIG. 18), and no pictogram is associated with other patients and icons “not set” are displayed.

For example, the display processor 220 may display a settable pictogram list when any of the pictograms (icons) is selected. For example, the pictogram list includes the multiple pictograms illustrated in FIG. 12A an 12B. In addition, the list mentioned here may include an icon corresponding to a reminder code. When a user performs an operation of selecting any of the pictograms in the list, the pictogram selected by the target patient and the pictogram code corresponding to the pictogram are assigned. This makes it possible to assign each patient with a pictogram code according to the patient's state. The pictogram code assigned here may be used for the processing of determining the recommended notification condition as has been described above using FIG. 10 to FIG. 14, or may be used for the processing of displaying the patient screen illustrated in FIG. 18.

### 4. Timing of Executing Each Processing

Next, a description will be given of a specific example of the timing of executing the processing of obtaining hospital ward load information (Step S101 of FIG. 5) and the timing of executing the processing of obtaining allowable load information (Step S102).

### 4.1 Hospital Ward Load Information

As has been described above, in the method of this embodiment, a load on a caregiver on per-hospital ward basis when notification processing is performed is obtained as hospital ward load information, and the relationship between the hospital ward load information and allowable load information is presented on a display screen such a setting screen.

As has been described above using FIG. 7, the hospital ward load information is obtained based on patient load information of f multiple patients. Accordingly, it is preferable that, when the patient load information changes, the hospital ward load information also changes according to this change. This makes it possible to inhibit the value of the hospital ward load information from deviating from the degree of loads that can be actually imposed on a caregiver. As a result, it is possible to realize more appropriate notification setting.

For example, the notification setting unit 210 may obtain patient load information based on an electronic medical record. Specifically, as has been described above using Step S303 of FIG. 7, the notification setting unit 210 may perform processing of reading disease state information, such as “limb disorder”, “brain infarction”, and “within Y days after operation”, from the electronic medical record and adding a value, corresponding to the disease state, to the patient load information. In addition, if the risks of falling down/falling off illustrated in No. 1 to 3 of FIG. 8 are described in the electronic medical record, these pieces of information may be read from the electronic medical record and used for processing of determining the patient load information.

In this case, the notification setting unit 210 may update the patient load information with an update of the electronic medical record as a trigger. For example, the notification setting unit 210 may periodically perform processing of reading an update history of the electronic medical record stored in the memory 250 and the like, and execute the processing in Step S101 of FIG. 5 if the electronic medical record is updated. The processing in Step S101 has been described in detail above using FIG. 7. Specifically, the notification setting unit 210 performs processing of adding a predetermined value to the value of the patient load information based on the result of reading the electronic medical record and load parameter master (Step S303 of FIG. 7). Alternatively, a push notification being an instruction to execute the processing of updating the patient load information may be sent to the notification setting unit 210 in response to an event where an operation of updating the electronic medical record is performed through the terminal device used by a doctor.

Then, the notification setting unit 210 updates hospital ward load information based on the updated patient load information (Step S304 of FIG. 7). Thereby, the patient load information and the hospital ward load information are updated promptly when a change in the disease state of the patient is recorded. Thus, it is possible to inhibit the value of the hospital ward load information from deviating from the degree of loads that can be actually imposed on a caregiver.

Meanwhile, in a case where a period of a given length is divided into multiple sub-periods, the notification setting unit 210 may perform processing of updating the hospital ward load information at a timing when any of the multiple sub-periods transitions to another sub-period. In other words, the notification setting unit 210 may perform processing of updating the hospital ward load information with the transition of the sub-period as a trigger.

The period of the given length mentioned here is one day, and the sub-periods may be periods obtained by dividing one day into multiple sections such as the morning, daytime, night, and midnight. This makes it possible to adjust the hospital ward load information appropriately during one day.

For example, many patients are expected to be sleeping at midnight. Accordingly, events that meet the notification conditions (such as bed departure) are less likely to occur at midnight as compared with other time slots when patients are active. Even when there is a patient by whom a large load can occur in the event of executing notification processing, no load will be imposed on a caregiver if no notification processing on this patient is executed. In other words, since the probability of occurrence of notification processing is low at midnight as compared with other time slots, the degree of loads that can occur is also relatively low. It is also conceivable that, because the movement of patients is small at midnight, even in a case where some sort of notification processing is executed, a caregiver can easily deal with the notification processing and its load is relatively small.

Accordingly, the notification setting unit 210 may correct the hospital ward load information according to the sub-period. For example, as described previously, since the probability of occurrence of notification processing and a load needed to deal with the notification processing are relatively low at midnight, the notification setting unit 210 may perform correction processing of reducing the value of the hospital ward load information. In addition, the correction processing mentioned here is not limited to that of reducing the value of the hospital ward load information. For example, the notification setting unit 210 may perform correction processing of increasing the value of the hospital ward load information in the daytime when patients are most active. Besides, various modifications can be made for the setting of time slots during one day and the contents of correction processing in each time slot.

Note that, at the time of updating the hospital ward load information, the value of the patient load information may be updated for each of multiple patients, and updated hospital ward load information may be obtained based on the pieces of patient load information thus updated. For example, the notification setting unit 210 may perform processing of multiplying the value of the patient load information by a given factor set for each patient. Alternatively, the memory 250 may store load master data that varies depending on time slots. Then, the notification setting unit 210 may obtain the patient load information in a time slot to be subjected to processing based on a pictogram code and a patient profile associated with a patient (disease state information) and the load master data that matches this time slot.

By updating the patient load information for each sub-period in this manner, it is possible to finely adjust the hospital ward load information. For example, in the case of using the load master data of FIG. 8, it is possible to adjust the correction range for each item in such a way as to increase the difference in the load value of the risk of falling down/falling off between the daytime and the midnight and decrease the difference in the distance from the nurse station between the daytime and the midnight. Accordingly, it is also possible to adjust the correction range of the patient load information flexibly according to which item of the load master data matches the patient.

Alternatively, when updating the hospital ward load information, the notification setting unit 210 may directly correct the hospital ward load information, obtained from the patient load information, without updating the value of the patient load information. For example, while the value of the hospital ward load information obtained from the patient load information is set as a reference value, the notification setting unit 210 may perform processing of multiplying the reference value by a correction factor that is set according to the time slot. In this case, it is not necessary to update individual patient load information, and thus possible to reduce processing load.

Meanwhile, the period of the given length and the sub-period are not limited to those of the above example. For example, the given period may be one week and the sub-period may be one day. In this case, it is possible to adjust the patient load information according to the days of the week, for example. For example, in a case where a care (such as rehabilitation) is provided in the hospital ward only on a specific day of the week and not provided on the other days of the week, it is conceivable that the probability of occurrence of notification processing and the degree of loads that can occur when the notification processing is performed are different between the specific day of the week and the other days of the week. In this respect, by adjusting the hospital ward load information according to the days of the week, it is possible to reflect the above variation, depending on the days of the week, on the hospital ward load information.

Meanwhile, the given period may be a relatively long period such as one year and the sub-period may be one month or a season such as spring, summer, autumn, and winter. For example, in the case of a season which is a seasonal flu season, a burden on a caregiver may increase as compared with the other seasons. For example, in a case where flu occurs in a hospital ward, prevention of infection is needed in addition to a normal care. In this case, by increasing the value of the hospital ward load information in winter relative to those in the other seasons, for example, it is possible to deal with such load variations depending on the seasons. Note that, as described previously, the hospital ward load information may be updated by updating the patient load information or by directly correcting the hospital ward load information.

### 4.2 Allowable Load Information

Meanwhile, in a case where a caregiver includes multiple caregivers and the multiple caregivers work in shifts, the notification setting unit 210 may update allowable load information at a timing when the multiple caregivers switch. As has been described above using FIG. 15 and FIG. 16, the allowable load information is determined depending on the number of caregivers, for example. Accordingly, by updating the allowable load information at the switch timing (shift switch timing) when the number of caregivers may vary, it is possible to obtain the allowable load information with high precision.

For example, in the case of employing a three-shift system in which caregivers work in three shifts obtained by dividing a 24-hour period into three shifts, the processing illustrated in Step S102 of FIG. 5 is executed at each of the switch timing between the day shift and the semi-night shift, the switch timing between the semi-night shift and the night shift, and the switch timing between the night shift and the day shift. Besides, another mode such as a two-shift system may be used as a work mode.

For example, in the case of obtaining the allowable load information in the day shift, the notification setting unit 210 may execute the processing including regression analysis illustrated in FIG. 16 by using day-shift history data for the past one month. Likewise, the allowable load information in the semi-night shift is obtained based on the past semi-night-shift history data, and the allowable load information in the night shift is obtained based on the past night-shift history data. This makes it possible to divide the history data on per-working time slot basis, and thus possible to obtain the allowable load information with high precision. Here, the notification setting unit 210 may have one common history data without dividing it on per-working time slot basis.

In the above example, for example, the processing of obtaining the allowable load information may be executed a predetermined number of times (e.g. three times) every day. In this case, data used as the history data is data for the past one month since the timing of the processing. In other words, the notification setting unit 210 may perform processing of obtaining the allowable load information every day while shifting the range of the history data by one day. This suppresses deviation between the target range of the history data and the timing of the processing, thus making it possible to obtain the allowable load information precisely.

However, the range of the history data used at the time of obtaining the allowable load information is not limited to the period since the timing of the processing. For example, the notification setting unit 210 may obtain the allowable load information based on the history data acquired in the same season one year ago. For example, the degree of allowable loads may decrease when caregivers are recruited or transferred because the number of inexperienced caregivers increases. In this case, by using the history data of the same season, it is possible to obtain the allowable load information appropriately.

4.3 how to Deal with Patient Outside Hospital

The description has been given above of the example where the patient load information is obtained for patients inside the hospital (hereinafter also referred to as hospitalized patients) and the hospital ward load information is obtained based on the patient load information on per-hospital ward basis. However, the method of the present disclosure is not limited to this, and information on patients outside the hospital may be used.

The patient mentioned here is a patient whose electronic medical record has been acquired by the information processor 200. Specifically, the patient mentioned here may be a patient outside the hospital who has attended or has been hospitalized in the hospital having adopted the service according to this embodiment and who is now under medical treatment at a location other than the hospital such as home or a care facility. In this case, the information processor 200 may determine the risk of falling down/falling off and disease state information (such as whether a limb disorder exists) by referring to the electronic medical record of the patient outside the hospital.

The notification setting unit 210 may obtain the patient load information of the patient outside the hospital based on the electronic medical record of the patient outside the hospital and load master data. The patient load information of the patient outside the hospital is information indicating the degree of loads needed to deal with the patient outside the hospital when this patient outside the hospital is transported to the hospital ward, for example. The transportation mentioned here, in a narrow sense, refers to emergency transportation, but may include a case where the patient visits the outpatient department by himself/herself.

The processing of obtaining the patient load information is as in the example of FIG. 7. However, in this modified example, the processing target in Step S301 is not limited to the bed 100 inside the hospital ward and is expanded to the patient outside the hospital.

The notification setting unit 210 performs processing of adding the patient load information of the patient outside the hospital to the hospital ward load information of the hospital ward corresponding to an emergency room and/or the hospital ward load information of the hospital ward corresponding to a department where the patient has attended in the past. This makes it possible to obtain the hospital ward load information including a load produced when the patient outside the hospital is transported to the target hospital ward. As a result, it is possible to execute appropriate notification processing in consideration of a case where the patient outside the hospital is transported.

Note that, when the patient outside the hospital is set as a target, the notification setting unit 210 may obtain the patient load information or the hospital ward load information by a method different from that used when the hospitalized patient is set as a target. For example, when the patient outside the hospital is set as a target, the notification setting unit 210 may perform correction processing changing the value of the patient load information from that of the hospitalized patient.

For example, in a case where the patient outside the hospital has fallen down or fallen off when trying to get up from the bed 100, it is conceivable that the family of the patient outside the hospital and the like first checks the state of the patient and, unless the patient is severely wounded, sees how things go without transporting the patient to the hospital. The same goes for a case where a symptom such as breathing disorder occurs, and care at the hospital is not needed as long as the symptom is mild enough that the family of the patient outside the hospital and the like can deal with and solve it. In other words, it is assumed that the case where the patient outside the hospital is transported to the hospital means the patient is in a serious state and a load needed to deal with the patient is high. In consideration of this point, it is preferable that the patient load information of the patient outside the hospital is set higher than the patient load information of the hospitalized patient.

Meanwhile, as described previously, a caregiver working in the hospital (such as a nurse) does not need to deal with the patient outside the hospital as long as the family and the like can deal with the patient. On the other hand, the caregiver working in the hospital needs to deal with an anomaly of the hospitalized patient. In consideration of this point, since the probability for the patient outside the hospital to need care by the nurse and the like is lower than that for the hospitalized patient, it is preferable that the patient load information of the patient outside the hospital is set lower than the patient load information of the hospitalized patient. Further, in the case of emergency transportation, a transportation destination is not fixed but is determined dynamically among multiple hospitals, capable of providing emergency medical care, according to their availability. For this reason, even when the patient has been hospitalized in or has attended a certain hospital, the patient is not necessarily transported to this hospital but may be transported to another hospital. Also, in consideration of this point, since the probability for the patient outside the hospital to need care in the hospital ward is lower than that for the hospitalized patient, it is preferable that the patient load information of the patient outside the hospital is set lower than the patient load information of the hospitalized patient.

For example, the notification setting unit 210 may set a correction factor β1 which is larger than 1 and a correction factor β2 which is smaller than 1. The notification setting unit 210 may obtain the patient load information of the patient outside the hospital by obtaining the patient load information using the same load master data as that for the hospitalized patient and then correcting this information by the correction factors β1 and β2. The correction factor β1 is a factor which is set in consideration of the point that, as described previously, the disease state may be serious if the patient outside the hospital is transported to the hospital. The correction factor β2 is a factor which is set in consideration of the point that, as described previously, the patient outside the hospital may not be transported to the target hospital ward even if an anomaly occurs. Any values can be set as specific values for the factors β1 and β2, and these values can be determined flexibly in consideration of their degree of influence. For example, the notification setting unit 210 may determine the correction factor β1 based on the past medical history, the medication history, and the like of the patient outside the hospital. In addition, the notification setting unit 210 may acquire the history of accepting an emergency patient for each of multiple emergency wards included in a target area and set the correction factor β2 based on this history of acceptance.

Further, as has been described above, the hospital to which the patient outside the hospital is to be transported is not fixed. Accordingly, the notification setting unit 210 may perform processing of adding the patient load information of the patient outside the hospital to the hospital ward load information for each of multiple hospitals to which the patient outside the hospital may be transported. In this event, the notification setting unit 210 may vary the correction factor β2 depending on the hospital. For example, the notification setting unit 210 may obtain the ratio of the number of patients accepted to the total number of emergency patients for each of the hospitals based on the past history, and use this ratio as the correction factor β2 of the target hospital.

Meanwhile, the description has been given above of the example where the correction processing is performed after using the common load master data for the hospitalized patient and the patient outside the hospital, but the description is not limited to this. For example, the notification setting unit 210 may obtain the patient load information of the patient outside the hospital by using load master data for the patient outside the hospital that is different from the load master data for the hospitalized patient described above. In the load master data for the patient outside the hospital, for example, “hospital (hospital ward)” is included as the addition condition, and load values different from one hospital to another may be added.

Meanwhile, the description has been given above of the example where the processing of obtaining the patient load information for the hospitalized patient is different from that for the patient outside the hospital, but the present disclosure is not limited to this. For example, as described previously, the correction processing may be executed after the hospital ward load information is obtained. For example, the notification setting unit 210 may obtain the hospital ward load information in consideration of the patient outside the hospital by subjecting the hospital ward load information, obtained by the same processing between the hospitalized patient and the patient outside the hospital, to correction processing according to the ratio of the number of patients outside the hospital to the total number of patients, the history of accepting an emergency patient in the target hospital ward, and the like.

The processing after obtaining the hospital ward load information is as in the above example. In other words, for each of the multiple hospitals (hospital wards), the notification processing based on the relationship between the hospital ward load information including the patient outside the hospital and the allowable load information of this hospital ward is executed.

### 5. Collection of Data Using Bed

Meanwhile, although the description has been given above of the example where the bed 100 and the terminal device 300 are provided as separate units, the method of this disclosure is not limited to this. For example, the bed 100 may be a nursing care bed capable of adjusting the angle and height of the surface of the section as described previously, and may include a controller (control box) provided with a processor that controls the same and a memory. This controller may also function as the terminal device 300 of this embodiment.

For example, the information processor 200 stores therein a table that associates the ID of the bed 100 with the patient, and the patient using the bed 100 is identified by this table. The bed 100 (terminal device 300) identifies the position of the bed 100 by communicating with a communication device that identifies a location. For example, the communication device may be a router or may be a wall unit (such as a smart plug connected to commercial power) that is disposed on a wall surface of a location such as a room.

By communication between the bed 100 (terminal device 300) and the device having a communication function (such as the detection device 400), data of each device is registered in a state where the patient is identified. Such a bed is described, for example, in U.S. patent application Ser. No. 17/360,382, filed on Jun. 28, 2021 and entitled “Monitoring of patient supports” and in U.S. patent application Ser. No. 17/883,071, filed on Aug. 8, 2022 and entitled “Hospital headwall communication system”. These patent applications are fully incorporated in the specification of the present application by reference.

This makes it possible to collect various kinds of information near the bed 100, determine whether the notification condition is met based on the information thus collected, and present the determination result, for example.

Note that, although this embodiment has been described in detail above, it will be readily understood by those skilled in the art that various modifications can be made without departing from the novel matters and effects of this embodiment. Accordingly, all of these modifications shall fall within the scope of this disclosure. For example, the term that is mentioned at least once in the specification or drawings with a different term that is a broader term or synonym may be replaced by that different term at any point in the specification or drawings. In addition, all combinations of this embodiment and the modifications shall fall within the scope of this disclosure. Further, the configuration and operation of the information processing system, the bed, the detection device, the sensing device, the information processor, the terminal device, and the like are not limited to those described in this embodiment, and various modifications can be made.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An information processing system comprising:
a controller configured to set a condition for executing a notification processing related to a patient to a caregiver, the controller being configured to execute the notification processing if the condition is satisfied; and
a display configured to display a setting screen used for setting of the notification processing by controlling the controller, wherein
the controller is configured to:
obtain a first information indicating a degree of loads needed for the caregiver to deal with the notification processing on per-hospital ward basis, if the notification processing is executed, and
obtain a second information indicating a degree of allowable loads, based on a third information indicating a target time in dealing with the notification processing and the number of the caregivers to deal with the notification processing, and
the display is configured to display information indicating relationship between the first information and the second information in the setting screen.

2. The information processing system according to claim 1, wherein
the controller is configured to:
obtain, for each of the plurality of patients, a fourth information indicating a degree of loads needed for the caregiver to deal with the notification processing on per-hospital ward basis, if the notification processing is executed, and
obtain the first information, indicating the degree of loads on the hospital ward, based on the fourth information corresponding to the plurality of patients.

3. The information processing system according to claim 2, wherein
the controller is configured to obtain the fourth information based on at least one of a risk of falling of the patient, a disease state of the patient, and information on a room of the patient.

4. The information processing system according to claim 3, wherein
the controller is configured to:
obtain the fourth information based on an electronic medical record of the patient,
update the fourth information if the electronic medical record of the patient is updated, and
update the first information based on the updated fourth information.

5. The information processing system according to claim 4, wherein
in a case where a period is divided into a plurality of sub-periods, the controller is configured to update the first information at a timing when the time has passed from a first sub-period to a second sub-period.

6. The information processing system according to claim 1, wherein
the controller is configured to determine, based on a comparison processing between the first information and the second information, whether to permit setting of the condition for executing the notification processing on the patient for whom the notification processing has not been set yet, and
the display is configured to display different kinds of the setting screen between a case where the controller determines to permit the setting of the condition and a case where the controller determines not to permit the setting of the condition.

7. The information processing system according to claim 1, wherein in a case where the caregiver includes a plurality of caregivers, the controller is configured to update the second information at a timing when at least one of caregivers is coming to work or leaving work.

8. The information processing system according to claim 4, wherein in a case where the caregiver includes a plurality of caregivers, the controller is configured to update the second information at a timing when at least one of caregivers is coming to work or leaving work.

9. The information processing system according to claim 1, wherein the controller is configured to determines the second information based on a history of the first information and a history of time information indicating time needed for the caregiver to deal with the notification processing.

10. The information processing system according to claim 4, wherein the controller is configured to determines the second information based on a history of the first information and a history of time information indicating time needed for the caregiver to deal with the notification processing.

11. The information processing system according to claim 1, wherein the controller is configured to obtain the condition recommended for the patient based on at least one of the disease state of the patient, a moving range for the patient, moving means of the patient, and a movable range of a bed recommended for the patient.

12. The information processing system according to claim 11, wherein the display is configured to display the condition recommended for the patient in the setting screen.

13. The information processing system according to claim 11, wherein the display is configured to display an object indicating warning in the setting screen if the condition recommended for the patient and the condition set for the patient are different.

14. The information processing system according to claim 2, wherein the controller is configured to determine a notification mode of the notification processing based on the fourth information.

15. An information processing method comprising:
performing processing of displaying, on a display, a setting screen used for setting of notification processing;
setting a condition for executing the notification processing related to a patient to a caregiver;
executing the notification processing if the condition is satisfied;
in the setting of the condition,
obtaining a first information indicating a degree of loads needed for the caregiver to deal with the notification processing on per-hospital ward basis, if the notification processing is executed, and
obtaining a second information, indicating a degree of allowable loads, based on a third information indicating a target time in dealing with the notification processing and the number of the caregivers to deal with the notification processing; and
in the processing of displaying the setting screen,
performing processing of displaying the setting screen including information indicating relationship between the first information and the second information.
